SEQUENCE LISTING (65 sequences)

SEQ ID NO: 1 - protein, 500 aa, synthetic construct
  REGION 1..500: note = RUBY
MTAIKMNTNGEGETQHILMIPFMAQGHLRPFLELAMFLYKRSHVIITLLTTPLNAGFLRHLLHHHSYSSSGIRIVELPFNSTNHGLPPGIENTDKLTLPLVVSLFHSTISLDPHLRDYISRHFSPARPPLCVIHDVFLGWVDQVAKDVGSTGVVFTTGGAYGTSAYVSIWNDLPHQNYSDDQEFPLPGFPENHKFRRSQLHRFLRYADGSDDWSKYFQPQLRQSMKSFGWLCNSVEEIETLGFSILRNYTKLPIWGIGPLIASPVQHSSSDNNSTGAEFVQWLSLKEPDSVLYISFGSQNTISPTQMMELAAGLESSEKPFLWVIRAPFGFDINEEMRPEWLPEGFEERMKVKKQGKLVYKLGPQLEILNHESIGGFLTHCGWNSILESLREGVPMLGWPLAAEQAYNLKYLEDEMGVAVELARGLEGEISKEKVKRIVEMILERNEGSKGWEMKNRAVEMGKKLKDAVNEEKELKGSSVKAIDDFLDAVMQAKLEPSLQ

SEQ ID NO: 2 - DNA, 1509 bp, synthetic construct
  misc_feature 1..1509: note = RUBY
  source 1..1509: mol_type = other DNA
atgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaacgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtga

SEQ ID NO: 3 - protein, 219 aa, synthetic construct
  REGION 1..219: note = eYGFPuv
MTTFKIESRIHGNLNGEKFELVGGGVGEEGRLEIEMKTKDKPLAFSPFLLTTCMGYGFYHFASFPKGIKNIYLHAATNGGYTNTRKEIYEDGGILEVNFRYTYEFNKIIGDVECIGHGFPSQSPIFKDTIVKSCPTVDLMLPMSGNIIASSYAYAFQLKDGSFYTAEVKNNIDFKNPIHESFSKSGPMFTHRRVEETLTKENLAIVEYQQVFNSAPRDM

SEQ ID NO: 4 - DNA, 660 bp, synthetic construct
  misc_feature 1..660: note = eYGFPuv
  source 1..660: mol_type = other DNA
atgacaaccttcaaaatcgagtcccggatccacggcaacctcaacggggagaagttcgagttggttggaggtggagtaggtgaggagggtcgcctcgagattgagatgaagactaaagataaaccactggcattctctcccttcctgctgaccacttgcatgggttacgggttctaccacttcgccagcttcccaaaggggattaagaacatctatcttcatgctgcaacgaacggaggttacaccaacaccaggaaggagatctatgaagacggcggcatcttggaggtcaacttccgttacacttacgagttcaacaagatcatcggtgacgtcgagtgcattggacatggattcccaagtcagagtccgatcttcaaggacacgatcgtgaagagttgtcccacggtggacctgatgttgccaatgtccgggaacatcatcgccagctcctacgcttacgccttccaactgaaggacggctctttctacacggcagaagtcaagaacaacatagacttcaagaatccaatccacgagtccttctcgaagtcagggcccatgttcacccacagacgtgtcgaggagactctcaccaaggagaaccttgccatagtggagtaccagcaggttttcaacagcgccccaagagacatgtag

SEQ ID NO: 5 - protein, 265 aa, synthetic construct
  REGION 1..265: note = Kanamycin Resistance
MGIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 6 - DNA, 798 bp, synthetic construct
  misc_feature 1..798: note = Kanamycin Resistance
  source 1..798: mol_type = other DNA
atggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 7 - protein, 341 aa, synthetic construct
  REGION 1..341: note = Hygromycin Resistance
MKKPELTATSVEKFLIEKFDSVSDLMQLSEGEESRAFSFDVGGRGYVLRVNSCADGFYKDRYVYRHFASAALPIPEVLDIGEFSESLTYCISRRSQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKK

SEQ ID NO: 8 - DNA, 1026 bp, synthetic construct
  misc_feature 1..1026: note = Hygromycin Resistance
  source 1..1026: mol_type = other DNA
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgttcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatag

SEQ ID NO: 9 - protein, 138 aa, synthetic construct
  REGION 1..138: note = NpuDNAE_Arabidopsis - full-codon-optimized
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNMIKIATRKYLGKQNVYDIGVERDHNFALKNGFIASN

SEQ ID NO: 10 - DNA, 414 bp, synthetic construct
  misc_feature 1..414: note = NpuDNAE_Arabidopsis - full-codon-optimized
  source 1..414: mol_type = other DNA
tgcttgtcctacgagaccgagattcttactgttgagtatggattgcttccaatcggcaagatcgtagagaaacgtattgagtgtacagtttattctgttgacaacaacggaaatatctatactcagcctgttgctcaatggcacgatagaggggaacaagaggttttcgagtattgccttgaggatggtagcctcattcgagcaacaaaagatcacaaatttatgacagtggatggtcagatgttacctatcgatgaaattttcgaaagggaattagacctcatgagagtggataacctccctaatatgattaagattgcgacccgtaagtacctggggaagcaaaacgtttacgatatcggtgtggagagagatcacaattttgctctgaaaaatggattcatagcttctaat

SEQ ID NO: 11 - protein, 231 aa, synthetic construct
  REGION 1..231: note = RUBY nL231:C232
MTAIKMNTNGEGETQHILMIPFMAQGHLRPFLELAMFLYKRSHVIITLLTTPLNAGFLRHLLHHHSYSSSGIRIVELPFNSTNHGLPPGIENTDKLTLPLVVSLFHSTISLDPHLRDYISRHFSPARPPLCVIHDVFLGWVDQVAKDVGSTGVVFTTGGAYGTSAYVSIWNDLPHQNYSDDQEFPLPGFPENHKFRRSQLHRFLRYADGSDDWSKYFQPQLRQSMKSFGWL

SEQ ID NO: 12 - DNA, 693 bp, synthetic construct
  misc_feature 1..693: note = RUBY nL231:C232
  source 1..693: mol_type = other DNA
atgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaacgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctc

SEQ ID NO: 13 - protein, 269 aa, synthetic construct
  REGION 1..269: note = RUBY cL231:C232
CNSVEEIETLGFSILRNYTKLPIWGIGPLIASPVQHSSSDNNSTGAEFVQWLSLKEPDSVLYISFGSQNTISPTQMMELAAGLESSEKPFLWVIRAPFGFDINEEMRPEWLPEGFEERMKVKKQGKLVYKLGPQLEILNHESIGGFLTHCGWNSILESLREGVPMLGWPLAAEQAYNLKYLEDEMGVAVELARGLEGEISKEKVKRIVEMILERNEGSKGWEMKNRAVEMGKKLKDAVNEEKELKGSSVKAIDDFLDAVMQAKLEPSLQ

SEQ ID NO: 14 - DNA, 816 bp, synthetic construct
  misc_feature 1..816: note = RUBY nL231:C232
  source 1..816: mol_type = other DNA
tgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtga

SEQ ID NO: 15 - protein, 52 aa, synthetic construct
  REGION 1..52: note = eYGFPuv nT52:C53
MTTFKIESRIHGNLNGEKFELVGGGVGEEGRLEIEMKTKDKPLAFSPFLLTT

SEQ ID NO: 16 - DNA, 156 bp, synthetic construct
  misc_feature 1..156: note = RUBY nL231:C232
  source 1..156: mol_type = other DNA
atgacaaccttcaaaatcgagtcccggatccacggcaacctcaacggggagaagttcgagttggttggaggtggagtaggtgaggagggtcgcctcgagattgagatgaagactaaagataaaccactggcattctctcccttcctgctgaccact

SEQ ID NO: 17 - protein, 167 aa, synthetic construct
  REGION 1..167: note = eYGFPuv nT52:C53
CMGYGFYHFASFPKGIKNIYLHAATNGGYTNTRKEIYEDGGILEVNFRYTYEFNKIIGDVECIGHGFPSQSPIFKDTIVKSCPTVDLMLPMSGNIIASSYAYAFQLKDGSFYTAEVKNNIDFKNPIHESFSKSGPMFTHRRVEETLTKENLAIVEYQQVFNSAPRDM

SEQ ID NO: 18 - DNA, 504 bp, synthetic construct
  misc_feature 1..504: note = eYGFPuv nT52:C53
  source 1..504: mol_type = other DNA
tgcatgggttacgggttctaccacttcgccagcttcccaaaggggattaagaacatctatcttcatgctgcaacgaacggaggttacaccaacaccaggaaggagatctatgaagacggcggcatcttggaggtcaacttccgttacacttacgagttcaacaagatcatcggtgacgtcgagtgcattggacatggattcccaagtcagagtccgatcttcaaggacacgatcgtgaagagttgtcccacggtggacctgatgttgccaatgtccgggaacatcatcgccagctcctacgcttacgccttccaactgaaggacggctctttctacacggcagaagtcaagaacaacatagacttcaagaatccaatccacgagtccttctcgaagtcagggcccatgttcacccacagacgtgtcgaggagactctcaccaaggagaaccttgccatagtggagtaccagcaggttttcaacagcgccccaagagacatgtag

SEQ ID NO: 19 - protein, 131 aa, synthetic construct
  REGION 1..131: note = Kanamycin nT131:C132
MGIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPAT

SEQ ID NO: 20 - DNA, 393 bp, synthetic construct
  misc_feature 1..393: note = Kanamycin nT131:C132
  source 1..393: mol_type = other DNA
atggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacc

SEQ ID NO: 21 - protein, 134 aa, synthetic construct
  REGION 1..134: note = Kanamycin cT131:C132
CPFDHQAKHRIERARTRMEAGLVDQDDLDEEHQGLAPAELFARLKARMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 22 - DNA, 405 bp, synthetic construct
  misc_feature 1..405: note = Kanamycin cT131:C132
  source 1..405: mol_type = other DNA
tgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 23 - protein, 192 aa, synthetic construct
  REGION 1..192: note = Kanamycin nA192:C193
MGIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGEDLVVTHGDA

SEQ ID NO: 24 - DNA, 576 bp, synthetic construct
  misc_feature 1..576: note = Kanamycin cT131:C132
  source 1..576: mol_type = other DNA
atggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcc

SEQ ID NO: 25 - protein, 73 aa, synthetic construct
  REGION 1..73: note = Kanamycin cT131:C132
CLPNIMVENGRFSGFIDCGRLGVADRYQDIALATRDIAEELGGEWADRFLVLYGIAAPDSQRIAFYRLLDEFF

SEQ ID NO: 26 - DNA, 222 bp, synthetic construct
  misc_feature 1..222: note = Kanamycin cT131:C132
  source 1..222: mol_type = other DNA
tgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 27 - protein, 52 aa, synthetic construct
  REGION 1..52: note = Hygromycin nS52:C53
MKKPELTATSVEKFLIEKFDSVSDLMQLSEGEESRAFSFDVGGRGYVLRVNS

SEQ ID NO: 28 - DNA, 156 bp, synthetic construct
  misc_feature 1..156: note = Hygromycin nS52:C53
  source 1..156: mol_type = other DNA
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagc

SEQ ID NO: 29 - protein, 289 aa, synthetic construct
  REGION 1..289: note = Hygromycin cS52:C53
CADGFYKDRYVYRHFASAALPIPEVLDIGEFSESLTYCISRRSQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKK

SEQ ID NO: 30 - DNA, 870 bp, synthetic construct
  misc_feature 1..870: note = Hygromycin cS52:C53
  source 1..870: mol_type = other DNA
tgcgccgatggtttctacaaagatcgttatgtttatcggcactttgcatcggccgcgctcccgattccggaagtgcttgacattggggagtttagcgagagcctgacctattgcatctcccgccgttcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatag

SEQ ID NO: 31 - protein, 89 aa, synthetic construct
  REGION 1..89: note = Hygromycin nY89:C90
MKKPELTATSVEKFLIEKFDSVSDLMQLSEGEESRAFSFDVGGRGYVLRVNSCADGFYKDRYVYRHFASAALPIPEVLDIGEFSESLTY

SEQ ID NO: 32 - DNA, 267 bp, synthetic construct
  misc_feature 1..267: note = Hygromycin nY89:C90
  source 1..267: mol_type = other DNA
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctat

SEQ ID NO: 33 - protein, 252 aa, synthetic construct
  REGION 1..252: note = Hygromycin cY89:C90
CISRRSQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKK

SEQ ID NO: 34 - DNA, 759 bp, synthetic construct
  misc_feature 1..759: note = Hygromycin cY89:C90
  source 1..759: mol_type = other DNA
tgcatctcccgccgttcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatag

SEQ ID NO: 35 - protein, 102 aa, synthetic construct
  REGION 1..102: note = NpuDNAE_Arabidopsis(n)-codon-optimized
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 36 - DNA, 306 bp, synthetic construct
  misc_feature 1..306: note = NpuDNAE_Arabidopsis(n)-codon-optimized
  source 1..306: mol_type = other DNA
tgcttgtcctacgagaccgagattcttactgttgagtatggattgcttccaatcggcaagatcgtagagaaacgtattgagtgtacagtttattctgttgacaacaacggaaatatctatactcagcctgttgctcaatggcacgatagaggggaacaagaggttttcgagtattgccttgaggatggtagcctcattcgagcaacaaaagatcacaaatttatgacagtggatggtcagatgttacctatcgatgaaattttcgaaagggaattagacctcatgagagtggataacctccctaat

SEQ ID NO: 37 - protein, 36 aa, synthetic construct
  REGION 1..36: note = NpuDNAE(c)_Arabidopsis-codon- optimized
MIKIATRKYLGKQNVYDIGVERDHNFALKNGFIASN

SEQ ID NO: 38 - DNA, 108 bp, synthetic construct
  misc_feature 1..108: note = NpuDNAE(c)_Arabidopsis-codon- optimized
  source 1..108: mol_type = other DNA
atgattaagattgcgacccgtaagtacctggggaagcaaaacgtttacgatatcggtgtggagagagatcacaattttgctctgaaaaatggattcatagcttctaat

SEQ ID NO: 39 - DNA, 16 bp, synthetic construct
  misc_feature 1..16: note = eYGFPuv Forward Primer
  source 1..16: mol_type = other DNA
cacggcaacctcaacg

SEQ ID NO: 40 - DNA, 17 bp, synthetic construct
  misc_feature 1..17: note = eYGFPuv Reverse Primer
  source 1..17: mol_type = other DNA
ctcgacacgtctgtggg

SEQ ID NO: 41 - DNA, 18 bp, synthetic construct
  misc_feature 1..18: note = RUBY Forward Primer
  source 1..18: mol_type = other DNA
cagagcttgcgagaaagg

SEQ ID NO: 42 - DNA, 19 bp, synthetic construct
  misc_feature 1..19: note = RUBY Reverse Primer
  source 1..19: mol_type = other DNA
ggcggaggtgaacttgtag

SEQ ID NO: 43 - DNA, 26 bp, synthetic construct
  misc_feature 1..26: note = 608_HSPT87
  source 1..26: mol_type = other DNA
gactaccaaaaaattctaagaaacag

SEQ ID NO: 44 - DNA, 47 bp, synthetic construct
  misc_feature 1..47: note = 624_cloning forward Primer
  source 1..47: mol_type = other DNA
tcatttggagagaacacgggggactctagaatggatcatgcgaccct

SEQ ID NO: 45 - DNA, 47 bp, synthetic construct
  misc_feature 1..47: note = 625_cloning Reverse Primer
  source 1..47: mol_type = other DNA
aggaaacagctatgacatgattacgaattcggggaaattcgagctgg

SEQ ID NO: 46 - DNA, 18 bp, synthetic construct
  misc_feature 1..18: note = 626_cloning forward Primer
  source 1..18: mol_type = other DNA
tagatgccacctgacgtc

SEQ ID NO: 47 - DNA, 18 bp, synthetic construct
  misc_feature 1..18: note = 627_cloning Reverse Primer
  source 1..18: mol_type = other DNA
atctgcgaaagctcgaga

SEQ ID NO: 48 - DNA, 19 bp, synthetic construct
  misc_feature 1..19: note = 643_cloning Reverse Primer
  source 1..19: mol_type = other DNA
ggcggaggtgaacttgtag

SEQ ID NO: 49 - DNA, 48 bp, synthetic construct
  misc_feature 1..48: note = 644_cloning Forward Primer
  source 1..48: mol_type = other DNA
cggctcctacaagttcacctccgcctgatagtgaatatgaagatgaag

SEQ ID NO: 50 - DNA, 19 bp, synthetic construct
  misc_feature 1..19: note = 645_cloning Forward Primer
  source 1..19: mol_type = other DNA
taatctggggacctgcagg

SEQ ID NO: 51 - DNA, 17 bp, synthetic construct
  misc_feature 1..17: note = 646_cloning Reverse Primer
  source 1..17: mol_type = other DNA
gtcaagagtcccccgtg

SEQ ID NO: 52 - DNA, 43 bp, synthetic construct
  misc_feature 1..43: note = 647_cloning Forward Primer
  source 1..43: mol_type = other DNA
ggagagaacacgggggactcttgacatgaccgccatcaagatg

SEQ ID NO: 53 - DNA, 20 bp, synthetic construct
  misc_feature 1..20: note = 649_cloning Forward Primer
  source 1..20: mol_type = other DNA
tgcttgtcctacgagaccga

SEQ ID NO: 54 - DNA, 57 bp, synthetic construct
  misc_feature 1..57: note = 650_cloning Reverse Primer
  source 1..57: mol_type = other DNA
gctttcctgggcaacgcttgttccacgcgttgtgtacagatatatgttgaattattg

SEQ ID NO: 55 - DNA, 44 bp, synthetic construct
  misc_feature 1..44: note = 651_cloning Reverse Primer
  source 1..44: mol_type = other DNA
gaatctcggtctcgtaggacaagcagagccagccaaaagacttc

SEQ ID NO: 56 - DNA, 48 bp, synthetic construct
  misc_feature 1..48: note = 654_cloning Forward Primer
  source 1..48: mol_type = other DNA
gctctgaaaaatggattcatagcttctaattgcaactccgtggaagag

SEQ ID NO: 57 - DNA, 27 bp, synthetic construct
  misc_feature 1..27: note = 656_cloning Reverse Primer
  source 1..27: mol_type = other DNA
cacaccaaatatttcatcttcatcttc

SEQ ID NO: 58 - DNA, 30 bp, synthetic construct
  misc_feature 1..30: note = 657_cloning Forward Primer
  source 1..30: mol_type = other DNA
gatatgaagatgaagatgaaatatttggtg

SEQ ID NO: 59 - DNA, 758 bp, synthetic construct
  misc_feature 1..758: note = GB104
  source 1..758: mol_type = other DNA
ctatctctctcgagctttcgcagatatggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcttgtcctacgagaccgagattcttactgttgagtatggattgcttccaatcggcaagatcgtagagaaacgtattgagtgtacagtttattctgttgacaacaacggaaatatctatactcagcctgttgctcaatggcacgatagaggggaacaagaggttttcgagtattgccttgaggatggtagcctcattcgagcaacaaaagatcacaaatttatgacagtggatggtcagatgttacctatcgatgaaattttcgaaagggaattagacctcatgagagtggataacctccctaattaggctcgagtttctccataataatgtgtgagta

SEQ ID NO: 60 - DNA, 606 bp, synthetic construct
  misc_feature 1..606: note = GB105
  source 1..606: mol_type = other DNA
gtctctctctacaaatctatctctctcgagatgattaagattgcgacccgtaagtacctggggaagcaaaacgtttacgatatcggtgtggagagagatcacaattttgctctgaaaaatggattcatagcttctaattgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaagtagatgccgaccggatctgtcgatcgacaagctcgagtttctccataataatgtgtgagta

SEQ ID NO: 61 - DNA, 141 bp, synthetic construct
  misc_feature 1..141: note = GB118
  source 1..141: mol_type = other DNA
acttttaatcaaatcaagattaaagttaattaaatgattaagattgcgacccgtaagtacctggggaagcaaaacgtttacgatatcggtgtggagagagatcacaattttgctctgaaaaatggattcatagcttctaat

SEQ ID NO: 62 - DNA, 780 bp, synthetic construct
  misc_feature 1..780: note = GB187
  source 1..780: mol_type = other DNA
cattctgcctggggacgtcggagcaagcttgatttaggtgacactatagaatacaagctacttgttctttttgcaggatccaccatggattataaggatgacgatgacaaagcagactacaaagacgacgatgataaggctgattataaagatgatgacgacaaaggccggccaatgaagaaaccggaactgactgcaacatctgtcgagaagtttctgattgaaaagttcgattctgtttccgatctcatgcagctcagtgagggcgaagagtccagagctttctcattcgacgtgggcgggcgcggatacgtactgcgagttaattcttgtgccgacggtttctacaaagatcggtatgtctatcggcatttcgcatctgccgcactgcctatcccggaggtgctggacataggtgaatttagtgaatcactgacttattgcctgagttatgagactgagattctgaccgtagaatatggtctgcttccaatcggcaagattgtggaaaaaaggatcgagtgtaccgtgtattctgtggacaataacggcaatatttacacccaacctgtggcacagtggcatgaccggggggagcaggaagtttttgaatactgcttggaggatggatccctcattagagctacaaaagatcacaagtttatgaccgtagacggacagatgttgccaatcgacgagatctttgagagggagctggatctgatgcgggtggacaatctgcccaactagtctagaactatagtgagtcgtattacgtag

SEQ ID NO: 63 - DNA, 669 bp, synthetic construct
  misc_feature 1..669: note = GB188
  source 1..669: mol_type = other DNA
cattctgcctggggacgtcggagcaagcttgatttaggtgacactatagaatacaagctacttgttctttttgcaggatccaccatggattataaggatgacgatgacaaagcagactacaaagacgacgatgataaggctgattataaagatgatgacgacaaaggccggccaatgaagaaaccggaactgactgcaacatctgtcgagaagtttctgattgaaaagttcgattctgtttccgatctcatgcagctcagtgagggcgaagagtccagagctttctcattcgacgtgggcgggcgcggatacgtactgcgagttaattcttgcctgagttatgagactgagattctgaccgtagaatatggtctgcttccaatcggcaagattgtggaaaaaaggatcgagtgtaccgtgtattctgtggacaataacggcaatatttacacccaacctgtggcacagtggcatgaccggggggagcaggaagtttttgaatactgcttggaggatggatccctcattagagctacaaaagatcacaagtttatgaccgtagacggacagatgttgccaatcgacgagatctttgagagggagctggatctgatgcgggtggacaatctgcccaactagtctagaactatagtgagtcgtattacgtag

SEQ ID NO: 64 - DNA, 1062 bp, synthetic construct
  misc_feature 1..1062: note = GB189
  source 1..1062: mol_type = other DNA
cattctgcctggggacgtcggagcaagcttgatttaggtgacactatagaatacaagctacttgttctttttgcaggatccaccatgatcaagatcgccacccggaaatacctcggcaaacagaacgtgtatgatatcggggtggagagggatcataacttcgccctcaaaaatgggtttattgcctctaattgcatctcccgcagaagccaaggcgtgactcttcaggaccttccagagactgaactccccgccgtcctgcagcctgttgctgaggccatggacgccatagccgccgccgacctgagccagacctctggttttgggcctttcggccctcagggaattggccagtacaccacctggagggactttatttgcgccatcgccgacccccatgtgtatcactggcagaccgttatggacgataccgtttccgcaagcgtggcccaagctctggatgagctgatgctgtgggcggaggattgtcccgaagtcaggcatcttgtccacgccgactttggatctaacaatgtcctgactgataacggcagaatcactgccgtaatcgattggtcagaggccatgttcggcgactctcagtacgaggtggctaacatcttcttttggagaccgtggttggcctgtatggaacagcaaacaagatacttcgagcgccggcacccggaactggccggatctccgaggctgagggcctacatgctgaggataggactggaccagctctatcagtccctggtggatggaaatttcgacgatgcggcttgggcccagggccggtgcgacgcaatagtgcggagcggagctgggacagtgggccggacccagattgcccggcgatctgcagctgtgtggactgatggctgtgttgaggtgctggctgacagcggcaatcggaggcccagtacgcgacctagagcaaaaaaatacccatacgatgttccagattacgcttatccttatgacgtacctgactatgcatacccttatgatgtaccagactacgcttagtctagaactatagtgagtcgtattacgtag

SEQ ID NO: 65 - DNA, 1173 bp, synthetic construct
  misc_feature 1..1173: note = GB190
  source 1..1173: mol_type = other DNA
cattctgcctggggacgtcggagcaagcttgatttaggtgacactatagaatacaagctacttgttctttttgcaggatccaccatgatcaagatcgccacccggaaatacctcggcaaacagaacgtgtatgatatcggggtggagagggatcataacttcgccctcaaaaatgggtttattgcctctaattgtgccgacggtttctacaaagatcggtatgtctatcggcatttcgcatctgccgcactgcctatcccggaggtgctggacataggtgaatttagtgaatcactgacttattgcatctcccgcagaagccaaggcgtgactcttcaggaccttccagagactgaactccccgccgtcctgcagcctgttgctgaggccatggacgccatagccgccgccgacctgagccagacctctggttttgggcctttcggccctcagggaattggccagtacaccacctggagggactttatttgcgccatcgccgacccccatgtgtatcactggcagaccgttatggacgataccgtttccgcaagcgtggcccaagctctggatgagctgatgctgtgggcggaggattgtcccgaagtcaggcatcttgtccacgccgactttggatctaacaatgtcctgactgataacggcagaatcactgccgtaatcgattggtcagaggccatgttcggcgactctcagtacgaggtggctaacatcttcttttggagaccgtggttggcctgtatggaacagcaaacaagatacttcgagcgccggcacccggaactggccggatctccgaggctgagggcctacatgctgaggataggactggaccagctctatcagtccctggtggatggaaatttcgacgatgcggcttgggcccagggccggtgcgacgcaatagtgcggagcggagctgggacagtgggccggacccagattgcccggcgatctgcagctgtgtggactgatggctgtgttgaggtgctggctgacagcggcaatcggaggcccagtacgcgacctagagcaaaaaaatacccatacgatgttccagattacgcttatccttatgacgtacctgactatgcatacccttatgatgtaccagactacgcttagtctagaactatagtgagtcgtattacgtag

(19) United States
(12) Patent Application Publication
(10) Pub. No.: US 2025/0154517 A1
(43) Pub. Date: May 15, 2025

(54) SYSTEM FOR PLANT CO-TRANSFORMATION AND METHODS OF USE

(71) Applicant: UT-Battelle, LLC, Oak Ridge, TN (US)

(72) Inventors: Xiaohan Yang, Oak Ridge, TN (US); Md Mahmudul Hassan, Oak Ridge, TN (US); Haiwei Lu, Oak Ridge, TN (US); Gerald A. Tuskan, Oak Ridge, TN (US); Guoliang Yuan, Richland, WA (US)

(21) Appl. No.: 18/891,003

(22) Filed: Sep. 20, 2024

Related U.S. Application Data
(60) Provisional application No. 63/539,831, filed on Sep. 22, 2023.

(51) Int. Cl. C12N 15/82

(52) U.S. Cl. CPC C12N 15/8212; C12N 15/8271

(57) ABSTRACT

The current disclosure relates to a split-intein-based gene-stacking system through split-selectable-marker-enabled co-transformation in Arabidopsis thaliana and poplar. The disclosure is also directed to methods of co-transforming plant cells, comprising delivering DNA vectors into a plant cell.

### CROSS REFERENCE TO RELATED APPLICATIONS

This application claims the benefit of U.S. Provisional Patent Application No. 63/539,831, filed Sep. 22, 2023, the contents of which is incorporated herein by reference in its entirety.

### STATEMENT REGARDING FEDERALLY SPONSORED RESEARCH

The United States Government has rights in this invention pursuant to contract no. DE-AC05-00OR22725 between the United States Department of Energy and UT-Battelle, LLC.

### INCORPORATION BY REFERENCE OF SEQUENCE LISTING

The Sequence Listing in an XML file, named as 43398_5012_02_SequenceListing.xml of 100 KB, created on Sep. 17, 2024, and submitted to the United States Patent and Trademark Office via Patent Center, is incorporated herein by reference.

### BACKGROUND

Synthetic metabolic engineering in plants relies on the introduction of a complete synthetic pathway into the target plant to create novel plant traits and produce value-added metabolites and therapeutic proteins. A complete synthetic pathway is typically encoded by multiple genes that involves multiple genetic parts and gene circuits. Multigene engineering therefore is becoming more and more important for plant synthetic biology research. Also, a lot of complex plant traits (e.g., yield) are controlled by multiple genes, and genetic improvement of such polygenic traits requires multi-gene stacking. Agrobacterium-mediated transformation is to date the most widely used method for plant genetic engineering due to its relatively high efficiency. Although some progress in Agrobacterium-mediated transformation of large DNA fragments required for multigene engineering in plants has been achieved, it has been reported that large genomic DNA fragments are not stable in Agrobacterium and T-DNA can be truncated at the left and/or right ends before being inserted into the plant genome. Thus, the effective transformations of tens of genes into a plant genome and consequent optimal control of gene expression remain to be improved in plant engineering. Current plant co-transformation approaches rely on at least two selectable gene markers. The concentrations of combined antibiotics need to be tested and adjusted carefully to achieve optimal transgenic selection effect. Also, there is a difference in selection efficacy between different selectable markers. For example, HygR works better (lower rate of false positives) than KanR in the genetic transformation of some poplar genotypes (Cseke et al. Plant Cell Reports 26, 1529-1538 (2007); and Fan et al. Scientific Reports 5, 12217 (2015)).

An intein is an intervening protein domain that excises itself post-translationally from the host protein and ligates together the flanking N- and C-terminal residues, called exteins, to form a native peptide bond (FIG. 1A). Naturally split inteins to date have enabled the development of numerous tools for both synthetic and biological applications by providing a rapid and bioorthogonal means to link two polypeptides. As such, we previously show that split intein, derived from NpuDnaE, can be used to reduce the size of CRISPR/Cas9 system by split Cas9 into multiple fragments and result in effective base editing in plants (Yuan, G. et al. ACS Synthetic Biology 11, 2513-2517 (2022)).

### SUMMARY

In some aspects, the present disclosure is directed to a split selectable marker system using split inteins to enable single-selectable-marker-gene dependent co-transformation in plants. The disclosure is also directed to methods of co-transforming plant cells, comprising delivering DNA vectors into a plant cell.

In one aspect, the present disclosure is directed to a split selectable marker system for plant co-transformation, the system comprising:
A) a first vector comprising:
   (i) from 5′ to 3′, and operably linked:
      a first promoter,
      a nucleotide sequence encoding an N-terminal fragment of a selectable marker protein;
      a nucleotide sequence encoding an N-terminal fragment of an intein; and
      a first terminator;
      wherein the nucleotide sequence encoding the N-terminal fragment of the selectable marker protein is linked, in frame, to the nucleotide sequence encoding the N-terminal fragment of the intein; and
   (ii) a first gene of interest, wherein the first gene of interest comprises a promoter, a coding sequence, and a terminator;
and
B) a second vector comprising:
   (i) from 5′ to 3′, and operably linked:
      a second promoter,
      a nucleotide sequence encoding a C-terminal fragment of the intein;
      a nucleotide sequence encoding a C-terminal fragment of the selectable marker protein; and
      a second terminator;
      wherein the nucleotide sequence encoding the C-terminal fragment of the intein is linked, in frame, to the nucleotide sequence encoding the C-terminal fragment of the selectable marker protein; and
   (ii) a second gene of interest, wherein the second gene of interest comprises a promoter, a coding sequence, and a terminator;
wherein upon expression in a plant cell, the N-terminal fragment and the C-terminal fragment of the intein join the N-terminal fragment and the C-terminal fragment of the selectable marker protein to form a peptide bond.

In some embodiments, the first promoter and the second promoter are each an inducible or constitutive promoter. In some embodiments, the selectable marker protein is a protein that produces a visible signal. In some embodiments, the visible signal is a red pigment or fluorescent signal. In some embodiments, the selectable marker protein is RUBY or eYGFPuv. In some embodiments, the selectable marker protein is RUBY, and the split between the N-terminal fragment and the C-terminal fragment of RUBY occurs at an amino acid position within the first 240 amino acids of SEQ ID NO: 1. In some embodiments, the selectable marker protein is RUBY, and the split between the N-terminal fragment and the C-terminal fragment of RUBY occurs at amino acid position L231:C232 of SEQ ID NO: 1. In some embodiments, the selectable marker protein is e YGFPuv, and the split between the N-terminal fragment and the C-terminal fragment of e YGFPuv occurs at an amino acid position within the first 75 amino acids of SEQ ID NO: 3. In some embodiments, the selectable marker protein is eYGFPuv, and the split between the N-terminal fragment and the C-terminal fragment of eYGFPuv occurs at amino acid position T52:C53 of SEQ ID NO: 3.

In some embodiments, the selected marker protein is a protein encoded by an antibiotic resistance gene. In some embodiments, the antibiotic resistance gene is a kanamycin or hygromycin resistance gene. In some embodiments, the selectable marker protein is a protein encoded by kanamycin resistance gene, and the split between the N-terminal fragment and the C-terminal fragment of the protein occurs at an amino acid position within the first 200 amino acids of SEQ ID NO: 5. In some embodiments, the selectable marker protein is a protein encoded by kanamycin resistance gene, and the split between the N-terminal fragment and the C-terminal fragment of the protein occurs at amino acid position T131:C132 or A192:C193 of SEQ ID NO: 5.

In some embodiments, the selectable marker protein is a protein encoded by hygromycin resistance gene, and the split between the N-terminal fragment and the C-terminal fragment of the protein occurs at an amino acid position within the first 100 amino acids of SEQ ID NO: 7. In some embodiments, the selectable marker protein is a protein encoded by hygromycin resistance gene, and the split between the N-terminal fragment and the C-terminal fragment of the protein occurs at amino acid position S52:C53 or Y89:C90 of SEQ ID NO: 7.

In some embodiments of the system, the intein is NpuDnaE. In some embodiments, the split between the N-terminal fragment and the C-terminal fragment of NpuDnaE occurs at amino acid position within the first 110 amino acids. In some embodiments, the split between the N-terminal fragment and the C-terminal fragment of NpuDnaE occurs at amino acid position N102:I103.

In some embodiments, the plant is an herbaceous or woody plant. In some embodiments, the herbaceous plant is selected from the group comprising Nicotiana, Arabidopsis thaliana, Brassica rapa, Glycine max, Nicotiana benthamiana, Oryza sativa, Solanum lycopersicum, Solanum tuberosum, Panicum virgatum, Sorghum bicolor, and Zea mays. In some embodiments, the woody plant is selected from the group comprising Citrus sinensis, Eucalyptus grandis, Malus domestica, Populus tremula x P. alba INRA 717-1B4, Prunus persica, Vitis vinifera.

Another aspect of the current disclosure is directed to a method of co-transforming plant cells, the method comprising delivering DNA vectors into a plant cell:
A) a first vector comprising:
   (i) from 5′ to 3′, and operably linked:
      a first promoter,
      a nucleotide sequence encoding an N-terminal fragment of a selectable marker protein;
      a nucleotide sequence encoding an N-terminal fragment of an intein; and
      a first terminator;
      wherein the nucleotide sequence encoding the N-terminal fragment of the selectable marker protein is linked, in frame, to the nucleotide sequence encoding the N-terminal fragment of the intein; and
   (ii) a first gene of interest, wherein the first gene of interest comprises a promoter, a coding sequence, and a terminator;
and
B) a second vector comprising:
   (i) from 5′ to 3′, and operably linked:
      a second promoter,
      a nucleotide sequence encoding a C-terminal fragment of the intein;
      a nucleotide sequence encoding a C-terminal fragment of the selectable marker protein; and
      a second terminator;
      wherein the nucleotide sequence encoding the C-terminal fragment of the intein is linked, in frame, to the nucleotide sequence encoding the C-terminal fragment of the selectable marker protein; and
   (ii) a second gene of interest, wherein the second gene of interest comprises a promoter, a coding sequence, and a terminator;
wherein upon expression in a plant cell, the N-terminal fragment and the C-terminal fragment of the intein join the N-terminal fragment and the C-terminal fragment of the selectable marker protein to form a peptide bond.

In some embodiments, the first promoter and the second promoter are each an inducible or constitutive promoter. In some embodiments, the selectable marker protein is a protein that produces a visible signal. In some embodiments, the visible signal is a red pigment or fluorescent signal. In some embodiments, the selectable marker protein is RUBY or eYGFPuv. In some embodiments, the selectable marker protein is RUBY, and the split between the N-terminal fragment and the C-terminal fragment of RUBY occurs at an amino acid position within the first 240 amino acids of SEQ ID NO: 1. In some embodiments, the selectable marker protein is RUBY, and the split between the N-terminal fragment and the C-terminal fragment of RUBY occurs at amino acid position L231:C232 of SEQ ID NO: 1. In some embodiments, the selectable marker protein is eYGFPuv, and the split between the N-terminal fragment and the C-terminal fragment of eYGFPuv occurs at an amino acid position within the first 75 amino acids of SEQ ID NO: 3. In some embodiments, the selectable marker protein is eYGFPuv, and the split between the N-terminal fragment and the C-terminal fragment of eYGFPuv occurs at amino acid position T52:C53 of SEQ ID NO: 3.

In some embodiments, the selected marker protein is a protein encoded by an antibiotic resistance gene. In some embodiments, the antibiotic resistance gene is a kanamycin or hygromycin resistance gene. In some embodiments, the selectable marker protein is a protein encoded by kanamycin resistance gene, and the split between the N-terminal fragment and the C-terminal fragment of the protein occurs at an amino acid position within the first 200 amino acids of SEQ ID NO: 5. In some embodiments, the selectable marker protein is a protein encoded by kanamycin resistance gene, and the split between the N-terminal fragment and the C-terminal fragment of the protein occurs at amino acid position T131:C132 or A192:C193 of SEQ ID NO: 5.

In some embodiments, the selectable marker protein is a protein encoded by hygromycin resistance gene, and the split between the N-terminal fragment and the C-terminal fragment of the protein occurs at an amino acid position within the first 100 amino acids of SEQ ID NO: 7. In some embodiments, the selectable marker protein is a protein encoded by hygromycin resistance gene, and the split between the N-terminal fragment and the C-terminal fragment of the protein occurs at amino acid position S52:C53 or Y89:C90 of SEQ ID NO: 7.

In some embodiments, the intein is NpuDnaE. In some embodiments, the split between the N-terminal fragment and the C-terminal fragment of NpuDnaE occurs at amino acid position within the first 110 amino acids. In some embodiments, the split between the N-terminal fragment and the C-terminal fragment of NpuDnaE occurs at amino acid position N102: I103.

In some embodiments, the plant is an herbaceous or woody plant. In some embodiments, the plant is an herbaceous or woody plant. In some embodiments, the herbaceous plant is selected from the group comprising Nicotiana, Arabidopsis thaliana, Brassica rapa, Glycine max, Nicotiana benthamiana, Oryza sativa, Solanum lycopersicum, Solanum tuberosum, Panicum virgatum, Sorghum bicolor, and Zea mays. In some embodiments, the woody plant is selected from the group comprising Citrus sinensis, Eucalyptus grandis, Malus domestica, Populus tremula x P. alba INRA 717-1B4, Prunus persica, Vitis vinifera.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1A-F. The NpuDnaE intein-mediated split selectable marker system. (A) Trans-splicing mechanism reaction by split inteins. (B) Illustration of vector design of a target gene. (C) Identification of potential split site of eYGFPuv. (D) Identification of potential split sites of gene Kan^{R} and Hyg^{R}. (E) Illustration of RUBY reporter transformation on left and eYGFPuv reporter transformation under UV light on right. (F) Experiment design of the split-selectable markers mediated co-transformation in plants.

FIG. 2A-G. Split RUBY and the split-selectable markers mediated gene stacking in tobacco and Arabidopsis. (A) Split selectable marker for co-selection of two separate transgenic vectors. (B) Illustration of RUBY reporter. (C) Identification of potential split site of gene GT. (D) Selection of T1 seedling on MS medium containing Kanamycin or Hygromycin. (E) Phenotyping of Kanamycin resistant T1 transformants (six weeks). (F) Phenotyping of Hygromycin resistant T1 transformants (four weeks). (G) Genotyping of T1 transformants using primers of eYGFPuv and RUBY, respectively.

FIG. 3A-B. Analysis of T2 plants after the split-selectable markers mediated gene stacking in Arabidopsis. (A) Selection of T2 seedlings on MS medium containing Kanamycin or Hygromycin. (B) Phenotyping of Kanamycin-resistant T2 transformants (5 weeks) and Hygromycin-resistant T2 transformants (4 weeks). Scale bar, 1 cm.

FIG. 4A-C. The split-selectable markers mediated gene stacking in poplar. (A) Root induction in root induction medium supplied with Hygromycin and phenotyping of transgenic events with/without UV light. Scale bar, 1 cm. (B) Genotyping of transgenic poplar events using primers of eYGFPuv and RUBY, respectively. (C) The analysis and alignment of genotyping and phenotyping of transgenic plants. The blue block represents positive events, while gray block represents negative events.

FIG. 5A-B. Western blot analysis of trans-splicing of the Hyg^{R} protein. (A) The N-terminal fragments of Hyg^{R} (F1 and F3) are N-terminally tagged with 3xFLAG epitope while the C-terminal fragments of Hyg^{R} (F2 and F4) are C-terminally tagged with 3xHA epitope. (B) Western blot was performed with the proteins extracted from human kidney cells, which were either transfected with the plasmids containing one of fragment F1 to F4, respectively, or co-transfected with F1+F2 containing plasmids and F3+F4 containing plasmids. Red, green, and yellow bands indicate FLAG, HA, and merged bands, respectively. Actin serves as an equal-loading control.

FIG. 6. Representative illustration of vectors used.

FIG. 7. Genotyping of T2 transformants using primers of eYGFPuv and RUBY, respectively. The genomic DNA of wild type (WT) Arabidopsis was used as a negative control while eYGFPuv and RUBY plasmids were used as positive control for eYGFPuv and RUBY, respectively. R indicates biological replicates.

FIG. 8. Illustration of split-RUBY vectors.

### DETAILED DESCRIPTION

The current disclosure relates to a split-intein-based gene-stacking system through split-selectable-marker-enabled co-transformation in Arabidopsis thaliana and poplar. The disclosure is also directed to methods of co-transforming plant cells, comprising delivering DNA vectors into a plant cell.

Current plant co-transformation approaches rely on at least two selectable gene markers. For this protocol, the concentrations of combined antibiotics need to be tested and adjusted carefully to achieve optimal transgenic selection effect. There is also a difference in selection efficacy between different selectable markers, such that Hyg^{R} works better (lower rate of false positives) than Kan^{R} in the genetic transformation of some poplar genotypes. For the first time in plants, this disclosure demonstrates that the systems of split-Kan^{R} and split-Hyg^{R} are effective for both in planta and plant tissue culture co-transformation in herbaceous and woody plants.

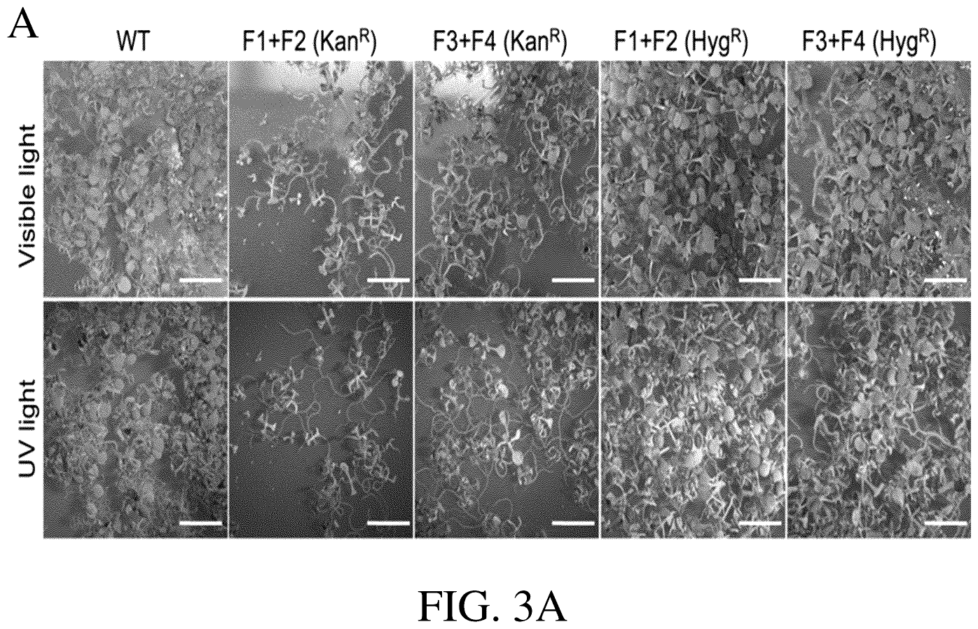

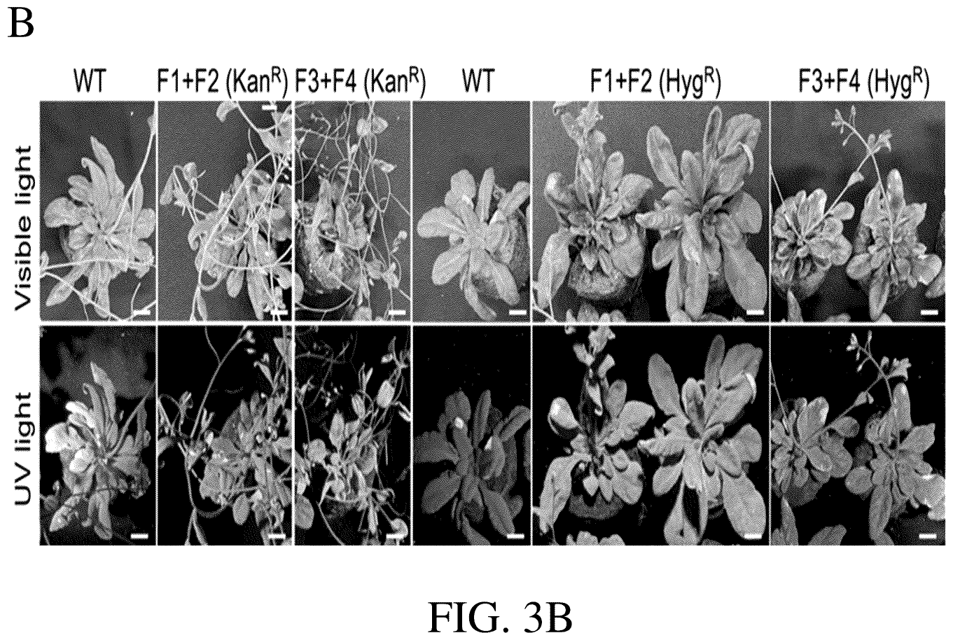

By dividing the larger cargoes across two T-DNAs, such systems enable the effective co-transformation of two separate binary vectors into a plant by Agrobacterium-mediated transformation. One constraint is that the insertion sites of the two T-DNAs are not controlled. Thus, the two T-DNAs will exhibit Mendelian segregation, as observed in FIG. 3A. In fact, most frequently, the offspring of crossings between different parents are used to study the inheritance of mutant traits in A. thaliana. For example, by mating the two single knockout mutants, constitutive double knockout Arabidopsis mutants lacking both DPE2 and PHS1 were produced. Yuan et al. generated a pp2ab′αβ double mutant by crossing two homozygous single-insertion mutants, pp2ab′α and pp2ab′β (Plant Physiol. 178, 317-328 (2018)). To develop homozygous drm1drm2 double mutant plants, Cao and Jacobsen crossed two isolated single mutants drm1 and drm2, in order to examine the function of the DRM genes. In general, a heterozygous double mutant will be generated in the F1 generation, which is equivalent to the T1 generation of this study, and a homozygous double mutant will be achieved in the F2 generation, which is equivalent to the T2 generation of this study. Therefore, a homozygous double mutant created by split-Kan^{R} or split-Hyg^{R} system is expected to be achieved in T2 generation for plant species with sexual reproduction, e.g., Arabidopsis, rice, and tomato. In contrast, for plants relying on vegetative propagation, e.g., poplar and citrus, the phenotype of double mutant will be inherited consistently without phenotype segregation.

The advantages of these co-transformation methods can reduce valuable time spent on constructing complex or long T-DNA molecules in binary vectors and sequential transformations, thus improving the capabilities for pathway engineering and genetic improvement of polygenic traits. In addition, the current common practice of expressing multiple genes involves the repeated use of the same or similar promoters due to the limited number of available promoters. Here, repetitive sequences within a plasmid can undergo intramolecular DNA recombination. This scenario is avoided with the use of the split selectable marker system described here. The choice of delivering multiple gene expression cassettes containing multiple identical sequences with two transformation vectors should allow a drastic reduction in the frequency of plasmid DNA recombination. Finally, this technology potentially doubles the capacity of existing transformation systems for multi-gene engineering in plants.

In one aspect, the present disclosure is directed to a split selectable marker system for plant co-transformation, the system comprising:
A) a first vector comprising:
   (i) from 5′ to 3′, and operably linked:
      a first promoter,
      a nucleotide sequence encoding an N-terminal fragment of a selectable marker protein;
      a nucleotide sequence encoding an N-terminal fragment of an intein; and
      a first terminator;
      wherein the nucleotide sequence encoding the N-terminal fragment of the selectable marker protein is linked, in frame, to the nucleotide sequence encoding the N-terminal fragment of the intein; and
   (ii) a first gene of interest, wherein the first gene of interest comprises a promoter, a coding sequence, and a terminator;
and
B) a second vector comprising:
   (i) from 5′ to 3′, and operably linked:
      a second promoter,
      a nucleotide sequence encoding a C-terminal fragment of the intein;
      a nucleotide sequence encoding a C-terminal fragment of the selectable marker protein; and
      a second terminator;
      wherein the nucleotide sequence encoding the C-terminal fragment of the intein is linked, in frame, to the nucleotide sequence encoding the C-terminal fragment of the selectable marker protein; and
   (ii) a second gene of interest, wherein the second gene of interest comprises a promoter, a coding sequence, and a terminator;
wherein upon expression in a plant cell, the N-terminal fragment and the C-terminal fragment of the intein join the N-terminal fragment and the C-terminal fragment of the selectable marker protein to form a peptide bond.

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. As used herein, the term “comprises” means “includes.” Thus, “comprising a nucleic acid molecule” means “including a nucleic acid molecule” without excluding other elements. It is further to be understood that any and all base sizes given for nucleic acids are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. All references, including patent applications and patents, are herein incorporated by reference in their entireties.

As used herein, the term “complementary” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “CRISPR” stands for “Clustered Regularly Interspaced Short Palindromic Repeats”. The CRISPR RNA array is a defining feature of CRISPR systems. The term “CRISPR” refers to the architecture of the array which includes constant direct repeats (DRs) interspaced with the variable spacers. Engineered CRISPR systems contain two components: a guide RNA (gRNA or sgRNA) and a CRISPR-associated endonuclease (Cas protein). The gRNA is a short synthetic RNA composed of a scaffold sequence necessary for Cas-binding and a user-defined ˜20 nucleotide spacer that defines the genomic target to be modified, i.e. a specific RNA sequence that recognizes the region of interest in the target DNA. Thus, one can change the genomic target of the Cas protein by simply changing the target sequence present in the gRNA.

As used herein, the term “restriction endonuclease recognition site” or “cut site” is intended to include, but is not limited to, a particular nucleic acid sequence to which one or more restriction enzymes bind, resulting in cleavage of a DNA molecule either at the restriction endonuclease recognition sequence itself, or at a sequence distal to the restriction endonuclease recognition sequence. Restriction enzymes include, but are not limited to, type I enzymes, type II enzymes, type IIS enzymes, type III enzymes and type IV enzymes. Additional exemplary enzymes include programmable nucleases such as Cas9, TALEN and ZFN as is known to those of skill in the art. The REBASE database provides a comprehensive database of information about restriction enzymes, DNA methyltransferases and related proteins involved in restriction-modification. It contains both published and unpublished work with information about restriction endonuclease recognition sites and restriction endonuclease cleavage sites, isoschizomers, commercial availability, crystal and sequence data (see Roberts et al. (2005) Nucl. Acids Res. 33: D230, incorporated herein by reference in its entirety for all purposes).

In certain aspects, primers of the present invention include one or more restriction endonuclease recognition sites that enable type IIS enzymes to cleave the nucleic acid several base pairs 3′ to the restriction endonuclease recognition sequence. As used herein, the term “type IIS” refers to a restriction enzyme that cuts at a site remote from its recognition sequence. Type IIS enzymes are known to cut at a known distance from their recognition sites ranging from 0 to 20 base pairs. Examples of Type IIs endonucleases include, but are not limited to, enzymes that produce a 3′ overhang, such as, for example, Bsr I, Bsm I, BstF5 I, BsrD I, Bts I, Mnl I, BciV I, Hph I, Mbo II, Eci I, Acu I, Bpm I, Mme I, BsaX I, Bcg I, Bae I, Bfi I, TspDT I, TspGW I, Taq II, Eco57 I, Eco57M I, Gsu I, Ppi I, and Psr I; enzymes that produce a 5′ overhang such as, for example, BsmA I, Ple I, Fau I, Sap I, BspM I, SfaN I, Hga I, Bvb I, Fok I, BceA I, BsmF I, Ksp632 I, Eco31 I, Esp3 I, Aar I; and enzymes that produce a blunt end, such as, for example, Mly I and Btr I. Type-IIs endonucleases are commercially available and are well known in the art (New England Biolabs, Beverly, Mass.). Information about the recognition sites, cut sites and conditions for digestion using type IIs endonucleases may be found, for example, on the Worldwide web at neb.com/nebecomm/enzymefindersearch bytypeIIs.asp). Restriction endonuclease sequences and restriction enzymes are well known in the art and restriction enzymes are commercially available (New England Biolabs, Ipswich, Mass.). Exemplary restriction enzymes include BtgZI, BsaI, sapI, aarl, and BsmBI and the like. One of skill will be readily able to identify other useful restriction enzymes from public information such as websites and periodicals based on the present disclosure such that an exhaustive list need not be presented here. In some embodiments, the restriction enzyme used is the same at the 5′ and 3′ ends of the nucleotide.

As used herein, “vector” refers to nucleic acid molecule into which a foreign nucleic acid molecule can be introduced without disrupting the ability of the vector to replicate and/or integrate in a host cell. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art.

A vector can include nucleic acid sequences that permit it to replicate in a host cell, such as an origin of replication. A vector can also include one or more selectable marker genes and other genetic elements known in the art. An integrating vector is capable of integrating itself into a host nucleic acid. An expression vector is a vector that contains the necessary regulatory sequences to allow transcription and translation of inserted gene or genes.

One type of vector is a “plasmid,” which refers to a circular double stranded DNA loop into which additional DNA segments can be inserted, such as by standard molecular cloning techniques. Another type of vector is a viral vector, wherein virally derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a tobacco mosaic virus (TMV), potato virus X (PVX), tobacco rattle virus (TRV), barley stripe mosaic virus (BSMV) or geminivirus vector. In some embodiments the geminiviral vector is a bean yellow dwarf virus vector or tomato yellow leaf curl virus.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

Certain vectors are capable of directing the expression of genes to which they are operatively linked. Such vectors are referred to herein as “expression vectors.” Common expression vectors are often in the form of plasmids. Recombinant expression vectors can comprise a nucleic acid provided herein (such as a guide RNA [which can be expressed from an RNA sequence or a RNA sequence], nucleic acid encoding a Cas protein, i.e. Cas9 or Cas12) in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Regulatory elements are contemplated for use with the methods and constructs described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector may comprise one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6, 7SK and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter and Pol II promoters described herein. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78 (3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Aspects of the methods, vectors and systems described herein may make use of terminator sequences. A terminator sequence includes a section of nucleic acid sequence that marks the end of a gene or operon in genomic DNA during transcription. This sequence mediates transcriptional termination by providing signals in the newly synthesized mRNA that trigger processes which release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art.

In one aspect, the present disclosure is directed to a split selectable marker system for plant co-transformation, the system comprising:
A) a first vector comprising:
   (i) from 5′ to 3′, and operably linked:
      a first promoter,
      a nucleotide sequence encoding an N-terminal fragment of a selectable marker protein;
      a nucleotide sequence encoding an N-terminal fragment of an intein; and
      a first terminator;
      wherein the nucleotide sequence encoding the N-terminal fragment of the selectable marker protein is linked, in frame, to the nucleotide sequence encoding the N-terminal fragment of the intein; and
   (ii) a first gene of interest, wherein the first gene of interest comprises a promoter, a coding sequence, and a terminator;
and
B) a second vector comprising:
   (i) from 5′ to 3′, and operably linked:
      a second promoter,
      a nucleotide sequence encoding a C-terminal fragment of the intein;
      a nucleotide sequence encoding a C-terminal fragment of the selectable marker protein; and
      a second terminator;
      wherein the nucleotide sequence encoding the C-terminal fragment of the intein is linked, in frame, to the nucleotide sequence encoding the C-terminal fragment of the selectable marker protein; and
   (ii) a second gene of interest, wherein the second gene of interest comprises a promoter, a coding sequence, and a terminator;
wherein upon expression in a plant cell, the N-terminal fragment and the C-terminal fragment of the intein join the N-terminal fragment and the C-terminal fragment of the selectable marker protein to form a peptide bond.

### Vectors

A vector is any nucleic acid that may be used as a vehicle to carry exogenous (foreign) genetic material into a cell. A vector, in some embodiments, is a DNA sequence that includes an insert (e.g., transgene) and a larger sequence that serves as the backbone of the vector. Non-limiting examples of vectors include plasmids, viruses/viral vectors, cosmids, and artificial chromosomes, any of which may be used as provided herein. In some embodiments, the vector is a viral vector, such as a viral particle. In some embodiments, the vector is an RNA-based vector, such as a self-replicating RNA vector. As described herein, a vector includes a promoter operably linked to a nucleic acid encoding a fragment of an intein and a fragment of selectable marker protein. In some embodiments, a vector also comprises a promoter operably linked to a nucleic acid, such as a transgene, encoding a molecule of interest.

The present disclosure is directed to a split selectable marker system using split inteins to enable single-selectable-marker-gene dependent co-transformation in plants. For example, to effect co-transformation of two genes of interest (GOIs), two vectors can be designed. In some embodiments, one vector (e.g., a first vector) comprises a first promoter, a nucleotide sequence encoding an N-terminal fragment of a selectable marker protein, a nucleotide sequence encoding an N-terminal fragment of an intein, a first terminator, and a first GOI. Additionally, the first promoter, nucleotide sequence encoding the N-terminal fragment of the selectable marker protein, the nucleotide sequence encoding the N-terminal fragment of the intein and the first terminator are operably linked, in frame, from 5′ to 3′. Another vector (e.g., a second vector) comprises a second promoter, a nucleotide sequence encoding a C-terminal fragment of the intein, a nucleotide sequence encoding a C-terminal fragment of the selectable marker protein, a second terminator, and a second GOI. The second promoter, the nucleotide sequence encoding the C-terminal fragment of the intein, the nucleotide sequence encoding the C-terminal fragment of the selectable marker protein, and the second terminator are operably linked, in frame, from 5′ to 3′. The nucleotide sequence encoding the N-terminal fragment of the selectable marker is operably linked, in frame, to the nucleotide sequence encoding the N-terminal fragment of the intein. Similarly, the nucleotide sequence encoding the C-terminal fragment of the intein is operably linked, in frame, to the nucleotide sequence encoding the C-terminal fragment of the selectable marker. Upon translation, the intein facilitates the adjoining of adjacent residues in the N-terminal and C-terminal fragments of the intein with a peptide bond, and during protein splicing, the intein removes itself from the protein, facilitating the joining of the adjacent peptides (i.e. the N-terminal and C-terminal fragments of the selectable marker) with a peptide bond. Thus, only when both vectors are successfully introduced into a single cell (hence both GOIs have also been introduced into the same cell), the full-length selectable marker is formed enabling selection of such a transfected cell.

In some embodiments, the first promoter and the second promoter are each an inducible promoter. In some embodiments, the first promoter and the second promoter are each a constitutive promoter. In some embodiments, the first promoter is an inducible promoter, and the second promoter is a constitutive promoter. In some embodiments, the first promoter is a constitutive promoter, and the second promoter is an inducible promoter.

### Inteins

An intein (intervening protein) carries out a unique auto-processing event known as protein splicing in which it excises itself out from a larger precursor polypeptide through the cleavage of two peptide bonds and, in the process, ligates the flanking extein (external protein) sequences through the formation of a new peptide bond. This rearrangement occurs post-translationally (or possibly co-translationally), as intein genes are found embedded in frame within other protein-coding genes. Furthermore, intein-mediated protein splicing is spontaneous; it requires no external factor or energy source, only the folding of the intein domain. In nature, the precursor protein contains three segments—an N-extein (N-terminal portion of the protein) followed by the intein followed by a C-extein (C-terminal portion of the protein). After splicing, the resulting protein contains the N-extein linked to the C-extein.

There are two types of inteins: cis-splicing inteins are single polypeptides that are embedded in a host protein, whereas trans-splicing inteins (referred to as split inteins) are separate polypeptides that mediate protein splicing after the intein pieces and their protein cargo associate (see, e.g., Paulus, H Annu Rev Biochem 69:447-496 (2000); and Saleh L, Perler F B Chem Rec 6:183-193 (2006)). Split inteins catalyze a series of chemical rearrangements that require the intein to be properly assembled and folded. The first step in splicing involves an N-S acyl shift in which the N-extein polypeptide is transferred to the side chain of the first residue of the intein. This is then followed by a trans-(thio) esterification reaction in which this acyl unit is transferred to the first residue of the C-extein (which is either serine, threonine, or cysteine) to form a branched intermediate. In the penultimate step of the process, this branched intermediate is cleaved from the intein by a transamidation reaction involving the C-terminal asparagine residue of the intein. This then sets up the final step of the process involving an S-N acyl transfer to create a normal peptide bond between the two exteins (Lockless, S W, Muir, T W PNAS 106 (27): 10999-11004 (2009)).

Split inteins are transcribed and translated as two separate polypeptides, the N-intein and C-intein, each fused to one extein. Upon translation, the intein fragments spontaneously and non-covalently assemble (cooperatively fold) into the canonical intein structure to carry out protein splicing in trans. The first two split inteins characterized, from the cyanobacteria Synechocystis species PCC6803 (Ssp) and Nostoc punctiforme PCC73102 (Npu), are orthologs naturally found inserted in the a subunit of DNA Polymerase III (DnaE). Npu is especially notable due its remarkably fast rate of protein trans-splicing (t½=50 s at 30° C.). The half-life of Npu is significantly shorter than that of Ssp (t½=80 min at 30° C.) (Shah, N H et al. J. Am. Chem. Soc. 135:5839 (2013)).

As used herein, split inteins catalyze the joining of two fragments (e.g., an N-terminal fragment and a C-terminal fragment) of a selectable marker protein, such as an antibiotic resistance protein or a fluorescent protein to produce a functional, full-length protein.

A split intein may be a natural split intein or an engineered split intein. Natural split inteins naturally occur in a variety of different organisms. The largest known family of split inteins is found within the DnaE genes of at least 20 cyanobacterial species (Caspi J, et al. Mol. Microbiol. 50:1569-1577 (2003)). Thus, in some embodiments of the present disclosure, a natural split intein is selected from DNA polymerase III (DnaE) inteins. Non-limiting examples of DnaE inteins include Synechocystis sp. DnaE (SspDnaE) inteins and Nostoc punctiforme (NpuDnaE) inteins.

In some embodiments, a split intein is an engineered split intein. Engineered split inteins may be produced from contiguous inteins (where a contiguous intein is artificially split) or may be modified natural split inteins that, for example, promote efficient protein purification, ligation, modification and cyclization (e.g., NpuGEP and CfaGEP, as described by Stevens, A J PNAS 114(32): 8538-8543 (2017)). Methods for engineering split inteins are described, for example, by Aranko, A S et al. Protein Eng Des Sel. 27 (8): 263-271 (2014), incorporated herein by reference. In some embodiments, the engineered split intein is engineered from DnaB inteins (Wu, H, et al. Biochim Biophys Acta 1387 (1-2): 422-432 (1998)). For example, the engineered split intein may be a SspDnaB S1 intein. In some embodiments, the engineered split intein is engineered from GyrB inteins. For example, the engineered split intein may be a SspGyrB S11 intein.

An N-terminal fragment of an intein may be any peptide fragment that includes the free amine group (—NH2) of the full-length protein, and a C-terminal fragment of an intein may be any peptide fragment that includes the free carboxyl group (—COOH), as long as the N-terminal and C-terminal fragments are capable of interacting with each other to fuse into the full intein. For example, in some embodiments, the amino acid sequence of the NpuDnaE intein is represented by SEQ ID NO: 9, as set forth in the following:

CLSYETEILTVEYGLLPIGKIVEKRIECTVYSVDNNGNIYTQPVAQWHD
RGEQEVFEYCLEDGSLIRATKDHKFMTVDGQMLPIDEIFERELDLMRVD
NLPNMIKIATRKYLGKQNVYDIGVERDHNFALKNGFIASN.

In some such embodiments, an N-terminal fragment of NpuDnaE comprises the amino acid sequence identified by SEQ ID NO: 35, while the C-terminal fragment of NpuDnaE comprises the amino acid sequence of SEQ ID NO: 37, wherein SEQ ID NO: 35 is:

CLSYETEILTVEYGLLPIGKIVEKRIECTVYSVDNNGNIYTQPVAQWHD
RGEQEVFEYCLEDGSLIRATKDHKFMTVDGQMLPIDEIFERELDLMRVD
NLPN

and wherein SEQ ID NO: 37 is:

MIKIATRKYLGKQNVYDIGVERDHNFALKNGFIASN.

When the N-terminal fragment and C-terminal fragment of NpuDnaE are represented by the amino acid sequences of SEQ ID NO: 35 and SEQ ID NO: 37, in some embodiments, the nucleotide sequences encoding these N-terminal and C-terminal fragments are set forth in SEQ ID NO: 36 and SEQ ID NO: 38, respectively.

In some embodiments of the disclosed system, the intein is NpuDnaE. In some embodiments, the NpuDnaE intein comprises an amino acid sequence having at least 90% sequence identity to the amino acid sequence of SEQ ID NO: 9. In some embodiments, the NpuDnaE intein comprises an amino acid sequence having at least 95% sequence identity to the amino acid sequence of SEQ ID NO: 9. In some embodiments, the NpuDnaE intein comprises an amino acid sequence having at least 96% sequence identity to the amino acid sequence of SEQ ID NO: 9. In some embodiments, the NpuDnaE intein comprises an amino acid sequence having at least 97% sequence identity to the amino acid sequence of SEQ ID NO: 9. In some embodiments, the NpuDnaE intein comprises an amino acid sequence having at least 98% sequence identity to the amino acid sequence of SEQ ID NO: 9. In some embodiments, the NpuDnaE intein comprises an amino acid sequence having at least 99% sequence identity to the amino acid sequence of SEQ ID NO: 9. In some embodiments, the NpuDnaE intein comprises the amino acid sequence of SEQ ID NO: 9.

In some embodiments, the split between the N-terminal fragment and the C-terminal fragment of an NpuDnaE intein occurs at an amino acid position within the first 110 amino acids of the NpuDnaE intein sequence. In some embodiments, the split between the N-terminal fragment and the C-terminal fragment of an NpuDnaE intein occurs at an amino acid position within the first 110 amino acids of a sequence having at least 90% sequence identity to SEQ ID NO: 9. In some embodiments, the split between the N-terminal fragment and the C-terminal fragment of an NpuDnaE intein occurs at an amino acid position within the first 110 amino acids of a sequence having at least 95% sequence identity to SEQ ID NO: 9. In some embodiments, the split between the N-terminal fragment and the C-terminal fragment of an NpuDnaE intein occurs at an amino acid position within the first 110 amino acids of a sequence having at least 96% sequence identity to SEQ ID NO: 9. In some embodiments, the split between the N-terminal fragment and the C-terminal fragment of an NpuDnaE intein occurs at an amino acid position within the first 110 amino acids of a sequence having at least 97% sequence identity to SEQ ID NO: 9. In some embodiments, the split between the N-terminal fragment and the C-terminal fragment of an NpuDnaE intein occurs at an amino acid position within the first 110 amino acids of a sequence having at least 98% sequence identity to SEQ ID NO: 9. In some embodiments, the split between the N-terminal fragment and the C-terminal fragment of an NpuDnaE intein occurs at an amino acid position within the first 110 amino acids of a sequence having at least 99% sequence identity to SEQ ID NO: 9. In some embodiments, the split between the N-terminal fragment and the C-terminal fragment of an NpuDnaE intein occurs at amino acid position N102:I103 of SEQ ID NO: 9.

In some embodiments, the N-terminal fragment of an NpuDnaE intein comprises an amino acid sequence having at least 90% sequence identity to the amino acid sequence of SEQ ID NO: 35. In some embodiments, the N-terminal fragment of an NpuDnaE intein comprises an amino acid sequence having at least 95% sequence identity to the amino acid sequence of SEQ ID NO: 35. In some embodiments, the N-terminal fragment of an NpuDnaE intein comprises an amino acid sequence having at least 96% sequence identity to the amino acid sequence of SEQ ID NO: 35. In some embodiments, the N-terminal fragment of an NpuDnaE intein comprises an amino acid sequence having at least 97% sequence identity to the amino acid sequence of SEQ ID NO: 35. In some embodiments, the N-terminal fragment of an NpuDnaE intein comprises an amino acid sequence having at least 98% sequence identity to the amino acid sequence of SEQ ID NO: 35. In some embodiments, the N-terminal fragment of an NpuDnaE intein comprises an amino acid sequence having at least 99% sequence identity to the amino acid sequence of SEQ ID NO: 35. In some embodiments, the N-terminal fragment of an NpuDnaE intein comprises the amino acid sequence of SEQ ID NO: 35.

In some embodiments, the C-terminal fragment of an NpuDnaE intein comprises an amino acid sequence having at least 90% sequence identity to the amino acid sequence of SEQ ID NO: 37. In some embodiments, the C-terminal fragment of an NpuDnaE intein comprises an amino acid sequence having at least 95% sequence identity to the amino acid sequence of SEQ ID NO: 37. In some embodiments, the C-terminal fragment of an NpuDnaE intein comprises an amino acid sequence having at least 96% sequence identity to the amino acid sequence of SEQ ID NO: 37. In some embodiments, the C-terminal fragment of an NpuDnaE intein comprises an amino acid sequence having at least 97% sequence identity to the amino acid sequence of SEQ ID NO: 37. In some embodiments, the C-terminal fragment of an NpuDnaE intein comprises an amino acid sequence having at least 98% sequence identity to the amino acid sequence of SEQ ID NO: 37. In some embodiments, the C-terminal fragment of an NpuDnaE intein comprises an amino acid sequence having at least 99% sequence identity to the amino acid sequence of SEQ ID NO: 37. In some embodiments the C-terminal fragment of an NpuDnaE intein comprises the amino acid sequence of SEQ ID NO: 37.

### Selectable Markers

Transgenic plant cells of the present disclosure are selected based on their expression of a full-length selectable marker protein. A selectable marker protein, generally, confers a trait suitable for artificial selection. Selectable marker proteins are well-known in the art. Non-limiting examples of selectable marker proteins include visible signal proteins and antibiotic resistance proteins.

Full-length selectable marker genes, in some embodiments, are produced by joining in the same cell two selectable marker gene fragments. In some embodiments, with reference to any full-length protein, one of the fragments is an N-terminal fragment (N-extein), while the other fragment is a C-terminal fragment (C-extein). Thus, in some embodiments, a first antibiotic resistance protein fragment is an N-terminal antibiotic resistance protein fragment, and a second antibiotic resistance protein fragment is a C-terminal antibiotic resistance protein fragment. In other embodiments, a first fluorescent protein fragment is an N-terminal fluorescent protein fragment, and a second fluorescent protein fragment is a C-terminal fluorescent protein fragment.

### Visible Signal Marker Proteins

In some embodiments, the selectable marker is a protein that produces a visible signal. In some embodiments, the visible signal is a red pigment or fluorescent signal. Fluorescent protein markers are commonly used in the art, especially in plant biology as they allow easy identification of transgenic events in plant transformation. Non-limiting examples of fluorescent proteins that may be used as provided herein include TagCFP, mTagCFP2, Czurite, ECFP2, mKalama1, Sirius, Sapphire, T-Sapphire, ECFP, Cerulean, SCFP3C, mTurquoise, mTurquoise2, monomeric Midoriishi-Cyan, TagCFP, mTFP1, EGFP, Emerald, Superfolder GFP, Monomeric Czami Green, TagGFP2, mUKG, mWasabi, Clover, mNeonGreen, EYFP, Citrine, Venus, SYFP2, TagYFP, Monomeric Kusabira-Orange, mKOK, mKO2, mOrange, mOrange2, mRaspberry, mCherry, mStrawberry, mScarlet, mTangerine, tdTomato, TagRFP, TagRFP-T, mCpple, mRuby, mRuby2, mPlum, HcRed-Tandem, mKate2, mNeptune, NirFP, TagRFP657, IFP1.4 and iRFP.

In some embodiments, RUBY and eYGFPuv are used as the selectable reporters which are visible to the naked eye under white and UV light, respectively, without a need for cost- and labor-intensive characterization. Indeed, the green fluorescence of plants expressing eYGFPuv can be observed consistently both in Arabidopsis and poplar. The red pigment of plants expressing RUBY, in contrast, was less consistent, particularly in poplar, where no typical RUBY phenotype was found. To address this issue, a more reliable reporter such as GUS or LUC tends to be a better option to replace the RUBY reporter.

In some embodiments, the selectable marker protein is a protein that produces a visible signal.

In some embodiments, the selectable marker is a protein that produces a red pigment. In some embodiments, the visible marker is RUBY, where the protein and the gene are referred to herein as a RUBY protein and a RUBY gene, respectively. In some embodiments, a RUBY protein comprises an amino acid sequence having at least 90% sequence identity to the amino acid sequence of SEQ ID NO: 1. In some embodiments, a RUBY protein comprises an amino acid sequence at least 95% sequence identity to the amino acid sequence of SEQ ID NO: 1. In some embodiments, a RUBY protein comprises an amino acid sequence having at least 96% sequence identity to the amino acid sequence of SEQ ID NO: 1. In some embodiments, a RUBY protein comprises an amino acid sequence having at least 97% sequence identity to the amino acid sequence of SEQ ID NO: 1. In some embodiments, a RUBY protein comprises an amino acid sequence having at least 98% sequence identity to the amino acid sequence of SEQ ID NO: 1. In some embodiments, a RUBY protein comprises an amino acid sequence having at least 99% sequence identity to the amino acid sequence of SEQ ID NO: 1. In some embodiments, a RUBY protein comprises the amino acid sequence of SEQ ID NO: 1, wherein SEQ ID NO: 1 is:

MTAIKMNTNGEGETQHILMIPFMAQGHLRPFLELAMFLYKRSHVIITLL
TTPLNAGFLRHLLHHHSYSSSGIRIVELPFNSTNHGLPPGIENTDKLTL
PLVVSLFHSTISLDPHLRDYISRHFSPARPPLCVIHDVFLGWVDQVAKD
VGSTGVVFTTGGAYGTSAYVSIWNDLPHQNYSDDQEFPLPGFPENHKFR
RSQLHRFLRYADGSDDWSKYFQPQLRQSMKSFGWLCNSVEEIETLGFSI
LRNYTKLPIWGIGPLIASPVQHSSSDNNSTGAEFVQWLSLKEPDSVLYI
SFGSQNTISPTQMMELAAGLESSEKPFLWVIRAPFGFDINEEMRPEWLP
EGFEERMKVKKQGKLVYKLGPQLEILNHESIGGFLTHCGWNSILESLRE
GVPMLGWPLAAEQAYNLKYLEDEMGVAVELARGLEGEISKEKVKRIVEM
ILERNEGSKGWEMKNRAVEMGKKLKDAVNEEKELKGSSVKAIDDFLDAV
MQAKLEPSLQ.

In some embodiments, the split between the N-terminal fragment and the C-terminal fragment of a RUBY protein occurs at an amino acid position within the first 240 amino acids. In some embodiments, the split between the N-terminal fragment and the C-terminal fragment of a RUBY protein occurs at an amino acid position within the first 240 amino acids of a sequence having at least 90% sequence identity to SEQ ID NO: 1. In some embodiments, the split between the N-terminal fragment and the C-terminal fragment of a RUBY protein occurs at an amino acid position within the first 240 amino acids of a sequence having at least 95% sequence identity to SEQ ID NO: 1. In some embodiments, the split between the N-terminal fragment and the C-terminal fragment of a RUBY protein occurs at an amino acid position within the first 240 amino acids of a sequence having at least 96% sequence identity to SEQ ID NO: 1. In some embodiments, the split between the N-terminal fragment and the C-terminal fragment of a RUBY protein occurs at an amino acid position within the first 240 amino acids of a sequence having at least 97% sequence identity to SEQ ID NO: 1. In some embodiments, the split between the N-terminal fragment and the C-terminal fragment of a RUBY protein occurs at an amino acid position within the first 240 amino acids of a sequence having at least 98% sequence identity to SEQ ID NO: 1. In some embodiments, the split between the N-terminal fragment and the C-terminal fragment of a RUBY protein occurs at an amino acid position within the first 240 amino acids of a sequence having at least 99% sequence identity to SEQ ID NO: 1. In some embodiments, the split between the N-terminal fragment and the C-terminal fragment of a RUBY protein occurs at amino acid position L231:C232 of SEQ ID NO: 1.

In some embodiments, the N-terminal fragment of a RUBY protein comprises an amino acid sequence having at least 90% sequence identity to the amino acid sequence of SEQ ID NO: 11. In some embodiments, the N-terminal fragment of a RUBY protein comprises an amino acid sequence having at least 95% sequence identity to the amino acid sequence of SEQ ID NO: 11. In some embodiments, the N-terminal fragment of a RUBY protein comprises an amino acid sequence having at least 96% sequence identity to the amino acid sequence of SEQ ID NO: 11. In some embodiments, the N-terminal fragment of a RUBY protein comprises an amino acid sequence having at least 97% sequence identity to the amino acid sequence of SEQ ID NO: 11. In some embodiments, the N-terminal fragment of a RUBY protein comprises an amino acid sequence having at least 98% sequence identity to the amino acid sequence of SEQ ID NO: 11. In some embodiments, the N-terminal fragment of a RUBY protein comprises an amino acid sequence having at least 99% sequence identity to the amino acid sequence of SEQ ID NO: 11. In some embodiments, the N-terminal fragment of a RUBY protein comprises the amino acid sequence of SEQ ID NO: 11, wherein SEQ ID NO: 11 is:

MTAIKMNTNGEGETQHILMIPFMAQGHLRPFLELAMFLYKRSHVIITLL
TTPLNAGFLRHLLHHHSYSSSGIRIVELPFNSTNHGLPPGIENTDKLTL
PLVVSLFHSTISLDPHLRDYISRHFSPARPPLCVIHDVFLGWVDQVAKD
VGSTGVVFTTGGAYGTSAYVSIWNDLPHQNYSDDQEFPLPGFPENHKFR
RSQLHRFLRYADGSDDWSKYFQPQLRQSMKSFGWL.

In some embodiments, the C-terminal fragment of a RUBY protein comprises an amino acid sequence having at least 90% sequence identity to the amino acid sequence of SEQ ID NO: 13. In some embodiments, the C-terminal fragment of a RUBY protein comprises an amino acid sequence having at least 95% sequence identity to the amino acid sequence of SEQ ID NO: 13. In some embodiments, the C-terminal fragment of a RUBY protein comprises an amino acid sequence having at least 96% sequence identity to the amino acid sequence of SEQ ID NO: 13. In some embodiments, the C-terminal fragment of a RUBY protein comprises an amino acid sequence having at least 97% sequence identity to the amino acid sequence of SEQ ID NO: 13. In some embodiments, the C-terminal fragment of a RUBY protein comprises an amino acid sequence having at least 98% sequence identity to the amino acid sequence of SEQ ID NO: 13. In some embodiments, the C-terminal fragment of a RUBY protein comprises an amino acid sequence having at least 99% sequence identity to the amino acid sequence of SEQ ID NO: 13. In some embodiments, the C-terminal fragment of a RUBY protein comprises the amino acid sequence of SEQ ID NO: 13, wherein SEQ ID NO: 13 is:

CNSVEEIETLGFSILRNYTKLPIWGIGPLIASPVQHSSSDNNSTGAEFVQ
WLSLKEPDSVLYISFGSQNTISPTQMMELAAGLESSEKPFLWVIRAPFGF
DINEEMRPEWLPEGFEERMKVKKQGKLVYKLGPQLEILNHESIGGFLTHC
GWNSILESLREGVPMLGWPLAAEQAYNLKYLEDEMGVAVELARGLEGEIS
KEKVKRIVEMILERNEGSKGWEMKNRAVEMGKKLKDAVNEEKELKGSSVK
AIDDFLDAVMQAKLEPSLQ.

In some embodiments, the visible signal is a fluorescent signal. In some embodiments, the visible marker protein is eYGFPuv, where the protein and the gene are referred to herein as a eYGFPuv protein and a eYGFPuv gene, respectively. In some embodiments, a eYGFPuv protein comprises an amino acid sequence having at least 90% sequence identity to the amino acid sequence of SEQ ID NO: 3. In some embodiments, a eYGFPuv protein comprises an amino acid sequence at least 95% sequence identity to the amino acid sequence of SEQ ID NO: 3. In some embodiments, a eYGFPuv protein comprises an amino acid sequence having at least 96% sequence identity to the amino acid sequence of SEQ ID NO: 3. In some embodiments, a eYGFPuv protein comprises an amino acid sequence having at least 97% sequence identity to the amino acid sequence of SEQ ID NO: 3. In some embodiments, a eYGFPuv protein comprises an amino acid sequence having at least 98% sequence identity to the amino acid sequence of SEQ ID NO: 3. In some embodiments, a eYGFPuv protein comprises an amino acid sequence having at least 99% sequence identity to the amino acid sequence of SEQ ID NO: 3. In some embodiments, a eYGFPuv protein comprises the amino acid sequence of SEQ ID NO: 3, wherein SEQ ID NO: 3 is:

MTTFKIESRIHGNLNGEKFELVGGGVGEEGRLEIEMKTKDKPLAFSPFLL
TTCMGYGFYHFASFPKGIKNIYLHAATNGGYTNTRKEIYEDGGILEVNFR
YTYEFNKIIGDVECIGHGFPSQSPIFKDTIVKSCPTVDLMLPMSGNIIAS
SYAYAFQLKDGSFYTAEVKNNIDFKNPIHESFSKSGPMFTHRRVEETLTK
ENLAIVEYQQVENSAPRDM.

In some embodiments, the split between the N-terminal fragment and the C-terminal fragment of a eYGFPuv protein occurs at an amino acid position within the first 75 amino acids. In some embodiments, the split between the N-terminal fragment and the C-terminal fragment of a eYGFPuv protein occurs at an amino acid position within the first 75 amino acids of a sequence having at least 90% sequence identity to SEQ ID NO: 3. In some embodiments, the split between the N-terminal fragment and the C-terminal fragment of a eYGFPuv protein occurs at an amino acid position within the first 75 amino acids of a sequence having at least 95% sequence identity to SEQ ID NO: 3. In some embodiments, the split between the N-terminal fragment and the C-terminal fragment of a eYGFPuv protein occurs at an amino acid position within the first 75 amino acids of a sequence having at least 96% sequence identity to SEQ ID NO: 3. In some embodiments, the split between the N-terminal fragment and the C-terminal fragment of a eYGFPuv protein occurs at an amino acid position within the first 75 amino acids of a sequence having at least 97% sequence identity to SEQ ID NO: 3. In some embodiments, the split between the N-terminal fragment and the C-terminal fragment of a eYGFPuv protein occurs at an amino acid position within the first 75 amino acids of a sequence having at least 98% sequence identity to SEQ ID NO: 3. In some embodiments, the split between the N-terminal fragment and the C-terminal fragment of a eYGFPuv protein occurs at an amino acid position within the first 75 amino acids of a sequence having at least 99% sequence identity to SEQ ID NO: 3. In some embodiments, the split between the N-terminal fragment and the C-terminal fragment of a eYGFPuv protein occurs at amino acid position T52:C53 of SEQ ID NO: 3.

In some embodiments, the N-terminal fragment of a eYGFPuv protein comprises an amino acid sequence having at least 90% sequence identity to the amino acid sequence of SEQ ID NO: 15. In some embodiments, the N-terminal fragment of a eYGFPuv protein comprises an amino acid sequence having at least 95% sequence identity to the amino acid sequence of SEQ ID NO: 15. In some embodiments, the N-terminal fragment of a eYGFPuv protein comprises an amino acid sequence having at least 96% sequence identity to the amino acid sequence of SEQ ID NO: 15. In some embodiments, the N-terminal fragment of a eYGFPuv protein comprises an amino acid sequence having at least 97% sequence identity to the amino acid sequence of SEQ ID NO: 15. In some embodiments, the N-terminal fragment of a eYGFPuv protein comprises an amino acid sequence having at least 98% sequence identity to the amino acid sequence of SEQ ID NO: 15. In some embodiments, the N-terminal fragment of a eYGFPuv protein comprises an amino acid sequence having at least 99% sequence identity to the amino acid sequence of SEQ ID NO: 15. In some embodiments, the N-terminal fragment of a eYGFPuv protein comprises the amino acid sequence of SEQ ID NO: 15, wherein SEQ ID NO: 15 is:

MTTFKIESRIHGNLNGEKFELVGGGVGEEGRLEIEMKTKDKPLAFSPFLL
TT.

In some embodiments, the C-terminal fragment of a eYGFPuv protein comprises an amino acid sequence having at least 90% sequence identity to the amino acid sequence of SEQ ID NO: 17. In some embodiments, the C-terminal fragment of an eYGFPuv protein comprises an amino acid sequence having at least 95% sequence identity to the amino acid sequence of SEQ ID NO: 17. In some embodiments, the C-terminal fragment of an eYGFPuv protein comprises an amino acid sequence having at least 96% sequence identity to the amino acid sequence of SEQ ID NO: 17. In some embodiments, the C-terminal fragment of an eYGFPuv protein comprises an amino acid sequence having at least 97% sequence identity to the amino acid sequence of SEQ ID NO: 17. In some embodiments, the C-terminal fragment of an eYGFPuv protein comprises an amino acid sequence having at least 98% sequence identity to the amino acid sequence of SEQ ID NO: 17. In some embodiments, the C-terminal fragment of an eYGFPuv protein comprises an amino acid sequence having at least 99% sequence identity to the amino acid sequence of SEQ ID NO: 17. In some embodiments, the C-terminal fragment of an eYGFPuv protein comprises the amino acid sequence of SEQ ID NO: 17, wherein SEQ ID NO: 17 is:

CMGYGFYHFASFPKGIKNIYLHAATNGGYTNTRKEIYEDGGILEVNFRYT
YEFNKIIGDVECIGHGFPSQSPIFKDTIVKSCPTVDLMLPMSGNIIASSY
AYAFQLKDGSFYTAEVKNNIDFKNPIHESFSKSGPMFTHRRVEETLTKEN
LAIVEYQQVENSAPRDM.

### Antibiotic Resistance Marker Proteins

In some embodiments, the selectable marker protein is a protein encoded by an antibiotic resistance gene. An antibiotic resistance gene is a gene encoding a protein that confers resistance to a particular antibiotic or class of antibiotics. Antibiotic resistance genes are well known in the art. Non-limiting examples of antibiotic resistance genes include those which encode proteins that confer resistance to hygromycin, G418, puromycin, phleomycin D1, blasticidin, kanamycin, spectinomycin, streptomycin, ampicillin, carbenicillin, bleomycin, erythromycin, polymyxin D, tetracycline and chloramphenicol. Any of which can be used as selectable marker genes in embodiments of the disclosure.

Kanamycin (also known as kanamycin A) is also an aminoglycoside bacteriocidal antibiotic. It is isolated from the bacterium Streptomyces kanamyceticus. Kanamycin kills a variety of bacteria by inducing mistranslation and indirectly inhibiting translocation during protein synthesis. The nptII gene produces an enzyme that inactivates kanamycin by transferring a phosphate group from ATP to kanamycin. nptII is a commonly used selection marker in bacteria, plants, and mammalian cells. Thus, in some embodiments of this disclosure, the selectable marker gene is the nptII gene.

In some embodiments, the antibiotic resistance gene is a kanamycin resistance gene. In some embodiments, the selectable marker protein is a protein that is encoded by a kanamycin resistance gene and is referred to herein as kanamycin resistance protein. In some embodiments, the selectable marker is a kanamycin resistance protein. In some embodiments, a kanamycin resistance protein comprises an amino acid sequence having at least 90% sequence identity to the amino acid sequence of SEQ ID NO: 5. In some embodiments, a kanamycin resistance protein comprises an amino acid sequence having at least 95% sequence identity to the amino acid sequence of SEQ ID NO: 5. In some embodiments, a kanamycin resistance protein comprises an amino acid sequence having at least 96% sequence identity to the amino acid sequence of SEQ ID NO: 5. In some embodiments, a kanamycin resistance protein comprises an amino acid sequence having at least 97% sequence identity to the amino acid sequence of SEQ ID NO: 5. In some embodiments, a kanamycin resistance protein comprises an amino acid sequence having at least 98% sequence identity to the amino acid sequence of SEQ ID NO: 5. In some embodiments, a kanamycin resistance protein comprises an amino acid sequence having at least 99% sequence identity to the amino acid sequence of SEQ ID NO: 5. In some embodiments, a kanamycin resistance protein comprises the amino acid sequence of SEQ ID NO: 5, wherein SEQ ID NO: 5 is:

MGIEQDGLHAGSPAAWVERLFGYDWAQQTIGCSDAAVFRLSAQGRPVLFV
KTDLSGALNELQDEAARLSWLATTGVPCAAVLDVVTEAGRDWLLLGEVPG
QDLLSSHLAPAEKVSIMADAMRRLHTLDPATCPFDHQAKHRIERARTRME
AGLVDQDDLDEEHQGLAPAELFARLKARMPDGEDLVVTHGDACLPNIMVE
NGRFSGFIDCGRLGVADRYQDIALATRDIAEELGGEWADRFLVLYGIAAP
DSQRIAFYRLLDEFF.

In some embodiments, the split between the N-terminal fragment and the C-terminal fragment of a kanamycin resistance protein occurs at an amino acid position within the first 200 amino acids of a sequence having at least 90% sequence identity to SEQ ID NO: 5. In some embodiments, the split between the N-terminal fragment and the C-terminal fragment of a kanamycin resistance protein occurs at an amino acid position within the first 200 amino acids of a sequence having at least 95% sequence identity to SEQ ID NO: 5. In some embodiments, the split between the N-terminal fragment and the C-terminal fragment of a kanamycin resistance protein occurs at an amino acid position within the first 200 amino acids of a sequence having at least 96% sequence identity to SEQ ID NO: 5. In some embodiments, the split between the N-terminal fragment and the C-terminal fragment of a kanamycin resistance protein occurs at an amino acid position within the first 200 amino acids of a sequence having at least 97% sequence identity to SEQ ID NO: 5. In some embodiments, the split between the N-terminal fragment and the C-terminal fragment of a kanamycin resistance protein occurs at an amino acid position within the first 200 amino acids of a sequence having at least 98% sequence identity to SEQ ID NO: 5. In some embodiments, the split between the N-terminal fragment and the C-terminal fragment of a kanamycin resistance protein occurs at an amino acid position within the first 200 amino acids of a sequence having at least 99% sequence identity to SEQ ID NO: 5. In some embodiments, the split between the N-terminal fragment and the C-terminal fragment of a kanamycin resistance protein occurs at amino acid position T131:C132 of SEQ ID NO: 5. In some embodiments, the split between the N-terminal fragment and the C-terminal fragment of a kanamycin resistance protein occurs at amino acid position A192:C193 of SEQ ID NO: 5.

In some embodiments, the N-terminal fragment of a kanamycin resistance protein comprises an amino acid sequence having at least 90% sequence identity to the amino acid sequence of SEQ ID NO: 19. In some embodiments, the N-terminal fragment of a kanamycin resistance protein comprises an amino acid sequence having at least 95% sequence identity to the amino acid sequence of SEQ ID NO: 19. In some embodiments, the N-terminal fragment of a kanamycin resistance protein comprises an amino acid sequence having at least 96% sequence identity to the amino acid sequence of SEQ ID NO: 19. In some embodiments, the N-terminal fragment of a kanamycin resistance protein comprises an amino acid sequence having at least 97% sequence identity to the amino acid sequence of SEQ ID NO: 19. In some embodiments, the N-terminal fragment of a kanamycin resistance protein comprises an amino acid sequence having at least 98% sequence identity to the amino acid sequence of SEQ ID NO: 19. In some embodiments, the N-terminal fragment of a kanamycin resistance protein comprises an amino acid sequence having at least 99% sequence identity to the amino acid sequence of SEQ ID NO: 19. In some embodiments, the N-terminal fragment of a kanamycin resistance protein comprises the amino acid sequence of SEQ ID NO: 19, wherein SEQ ID NO: 19 is:

MGIEQDGLHAGSPAAWVERLFGYDWAQQTIGCSDAAVFRLSAQGRPVLFV
KTDLSGALNELQDEAARLSWLATTGVPCAAVLDVVTEAGRDWLLLGEVPG
QDLLSSHLAPAEKVSIMADAMRRLHTLDPAT.

In some embodiments, the N-terminal fragment of a kanamycin resistance protein comprises an amino acid sequence having at least 90% sequence identity to the amino acid sequence of SEQ ID NO: 23. In some embodiments, the N-terminal fragment of a kanamycin resistance protein comprises an amino acid sequence having at least 95% sequence identity to the amino acid sequence of SEQ ID NO: 23. In some embodiments, the N-terminal fragment of a kanamycin resistance protein comprises an amino acid sequence having at least 96% sequence identity to the amino acid sequence of SEQ ID NO: 23. In some embodiments, the N-terminal fragment of a kanamycin resistance protein comprises an amino acid sequence having at least 97% sequence identity to the amino acid sequence of SEQ ID NO: 23. In some embodiments, the N-terminal fragment of a kanamycin resistance protein comprises an amino acid sequence having at least 98% sequence identity to the amino acid sequence of SEQ ID NO: 23. In some embodiments, the N-terminal fragment of a kanamycin resistance protein comprises an amino acid sequence having at least 99% sequence identity to the amino acid sequence of SEQ ID NO: 23. In some embodiments, the N-terminal fragment of a kanamycin resistance protein comprises the amino acid sequence of SEQ ID NO: 23, wherein SEQ ID NO: 23 is:

MGIEQDGLHAGSPAAWVERLFGYDWAQQTIGCSDAAVFRLSAQGRPVLFV
KTDLSGALNELQDEAARLSWLATTGVPCAAVLDVVTEAGRDWLLLGEVPG
QDLLSSHLAPAEKVSIMADAMRRLHTLDPATCPFDHQAKHRIERARTRME
AGLVDQDDLDEEHQGLAPAELFARLKARMPDGEDLVVTHGDA.

In some embodiments, the C-terminal of a kanamycin resistance protein comprises an amino acid sequence having at least 90% sequence identity to the amino acid sequence of SEQ ID NO: 21. In some embodiments, the C-terminal of a kanamycin resistance protein comprises an amino acid sequence having at least 95% sequence identity to the amino acid sequence of SEQ ID NO: 21. In some embodiments, the C-terminal of a kanamycin resistance protein comprises an amino acid sequence having at least 96% sequence identity to the amino acid sequence of SEQ ID NO: 21. In some embodiments, the C-terminal of a kanamycin resistance protein comprises an amino acid sequence having at least 97% sequence identity to the amino acid sequence of SEQ ID NO: 21. In some embodiments, the C-terminal of a kanamycin resistance protein comprises an amino acid sequence having at least 98% sequence identity to the amino acid sequence of SEQ ID NO: 21. In some embodiments, the C-terminal of a kanamycin resistance protein comprises an amino acid sequence having at least 99% sequence identity to the amino acid sequence of SEQ ID NO: 21. In some embodiments, the C-terminal of a kanamycin resistance protein comprises the amino acid sequence of SEQ ID NO: 21, wherein SEQ ID NO: 21 is:

CPFDHQAKHRIERARTRMEAGLVDQDDLDEEHQGLAPAELFARLKARMPD
GEDLVVTHGDACLPNIMVENGRFSGFIDCGRLGVADRYQDIALATRDIAE
ELGGEWADRFLVLYGIAAPDSQRIAFYRLLDEFF.

In some embodiments, the C-terminal of a kanamycin resistance protein comprises an amino acid sequence having at least 90% sequence identity to the amino acid sequence of SEQ ID NO: 25. In some embodiments, the C-terminal of a kanamycin resistance protein comprises an amino acid sequence having at least 95% sequence identity to the amino acid sequence of SEQ ID NO: 25. In some embodiments, the C-terminal of a kanamycin resistance protein comprises an amino acid sequence having at least 96% sequence identity to the amino acid sequence of SEQ ID NO: 25. In some embodiments, the C-terminal of a kanamycin resistance protein comprises an amino acid sequence having at least 97% sequence identity to the amino acid sequence of SEQ ID NO: 25. In some embodiments, the C-terminal of a kanamycin resistance protein comprises an amino acid sequence having at least 98% sequence identity to the amino acid sequence of SEQ ID NO: 25. In some embodiments, the C-terminal of a kanamycin resistance protein comprises an amino acid sequence having at least 99% sequence identity to the amino acid sequence of SEQ ID NO: 25. In some embodiments, the C-terminal of a kanamycin resistance protein comprises the amino acid sequence of SEQ ID NO: 25, wherein SEQ ID NO: 25 is:

CLPNIMVENGRFSGFIDCGRLGVADRYQDIALATRDIAEELGGEWADRFL
VLYGIAAPDSQRIAFYRLLDEFF.

Hygromycin (also known as Hygromycin B) is an antibiotic produced by the bacterium Streptomyces hygroscopicus. It is an aminoglycoside that kills bacteria, fungi and higher eukaryotic cells by inhibiting protein synthesis. Hygromycin phosphotransferase (HPT), encoded by the hpt gene (also referred to as the hph or aphIV gene) originally derived from Escherichia coli, detoxifies the aminocyclitol antibiotic hygromycin B. Thus, in some embodiments, the selectable marker gene of the present disclosure is the hpt gene.

In some embodiments, the antibiotic resistance gene is a hygromycin resistance gene. In some embodiments, the selectable marker protein is a protein that is encoded by a hygromycin resistance gene and is referred to herein as hygromycin resistance protein. In some embodiments, the selectable marker is a hygromycin resistance protein. In some embodiments, a hygromycin resistance protein comprises an amino acid sequence having at least 90% sequence identity to the amino acid sequence of SEQ ID NO: 7. In some embodiments, a hygromycin resistance protein comprises an amino acid sequence at least 95% sequence identity to the amino acid sequence of SEQ ID NO: 7. In some embodiments, a hygromycin resistance protein comprises an amino acid sequence having at least 96% sequence identity to the amino acid sequence of SEQ ID NO: 7. In some embodiments, a hygromycin resistance protein comprises an amino acid sequence having at least 97% sequence identity to the amino acid sequence of SEQ ID NO: 7. In some embodiments, a hygromycin resistance protein comprises an amino acid sequence having at least 98% sequence identity to the amino acid sequence of SEQ ID NO: 7. In some embodiments, a hygromycin resistance protein comprises an amino acid sequence having at least 99% sequence identity to the amino acid sequence of SEQ ID NO: 7. In some embodiments, a hygromycin resistance protein comprises the amino acid sequence of SEQ ID NO: 7, wherein SEQ ID NO: 7 is:

MKKPELTATSVEKFLIEKFDSVSDLMQLSEGEESRAFSFDVGGRGYVLRV
NSCADGFYKDRYVYRHFASAALPIPEVLDIGEFSESLTYCISRRAQGVTL
QDLPETELPAVLQPVAEAMDAIAAADLSQTSGFGPFGPQGIGQYTTWRDF
ICAIADPHVYHWQTVMDDTVSASVAQALDELMLWAEDCPEVRHLVHADFG
SNNVLTDNGRITAVIDWSEAMFGDSQYEVANIFFWRPWLACMEQQTRYFE
RRHPELAGSPRLRAYMLRIGLDQLYQSLVDGNFDDAAWAQGRCDAIVRSG
AGTVGRTQIARRSAAVWTDGCVEVLADSGNRRPSTPRAKE.

In some embodiments, the split between the N-terminal fragment and the C-terminal fragment of a hygromycin resistance protein occurs at an amino acid position within the first 100 amino acids of a sequence having at least 90% sequence identity to SEQ ID NO: 7. In some embodiments, the split between the N-terminal fragment and the C-terminal fragment of a hygromycin resistance protein occurs at an amino acid position within the first 100 amino acids of a sequence having at least 95% sequence identity to SEQ ID NO: 7. In some embodiments, the split between the N-terminal fragment and the C-terminal fragment of a hygromycin resistance protein occurs at an amino acid position within the first 100 amino acids of a sequence having at least 96% sequence identity to SEQ ID NO: 7. In some embodiments, the split between the N-terminal fragment and the C-terminal fragment of a hygromycin resistance protein occurs at an amino acid position within the first 100 amino acids of a sequence having at least 97% sequence identity to SEQ ID NO: 7. In some embodiments, the split between the N-terminal fragment and the C-terminal fragment of a hygromycin resistance protein occurs at an amino acid position within the first 100 amino acids of a sequence having at least 98% sequence identity to SEQ ID NO: 7. In some embodiments, the split between the N-terminal fragment and the C-terminal fragment of a hygromycin resistance protein occurs at an amino acid position within the first 100 amino acids of a sequence having at least 99% sequence identity to SEQ ID NO: 7. In some embodiments, the split between the N-terminal fragment and the C-terminal fragment of a hygromycin resistance protein occurs at amino acid position S52:C53 of SEQ ID NO: 7. In some embodiments, the split between the N-terminal fragment and the C-terminal fragment of a hygromycin resistance protein occurs at amino acid position Y89:C90 of SEQ ID NO: 7.

In some embodiments, the N-terminal fragment of a hygromycin resistance protein comprises an amino acid sequence having at least 90% sequence identity to the amino acid sequence of SEQ ID NO: 27. In some embodiments, the N-terminal fragment of a hygromycin resistance protein comprises an amino acid sequence having at least 95% sequence identity to the amino acid sequence of SEQ ID NO: 27. In some embodiments, the N-terminal fragment of a hygromycin resistance protein comprises an amino acid sequence having at least 96% sequence identity to the amino acid sequence of SEQ ID NO: 27. In some embodiments, the N-terminal fragment of a hygromycin resistance protein comprises an amino acid sequence having at least 97% sequence identity to the amino acid sequence of SEQ ID NO: 27. In some embodiments, the N-terminal fragment of a hygromycin resistance protein comprises an amino acid sequence having at least 98% sequence identity to the amino acid sequence of SEQ ID NO: 27. In some embodiments, the N-terminal fragment of a hygromycin resistance protein comprises an amino acid sequence having at least 99% sequence identity to the amino acid sequence of SEQ ID NO: 27. In some embodiments, the N-terminal fragment of a hygromycin resistance protein comprises the amino acid sequence of SEQ ID NO: 27, wherein SEQ ID NO: 27 is:

MKKPELTATSVEKFLIEKFDSVSDLMQLSEGEESRAFSFDVGGRGYVLRV
NS.

In some embodiments, the N-terminal fragment of a hygromycin resistance protein comprises an amino acid sequence having at least 90% sequence identity to the amino acid sequence of SEQ ID NO: 31. In some embodiments, the N-terminal fragment of a hygromycin resistance protein comprises an amino acid sequence having at least 95% sequence identity to the amino acid sequence of SEQ ID NO: 31. In some embodiments, the N-terminal fragment of a hygromycin resistance protein comprises an amino acid sequence having at least 96% sequence identity to the amino acid sequence of SEQ ID NO: 31. In some embodiments, the N-terminal fragment of a hygromycin resistance protein comprises an amino acid sequence having at least 97% sequence identity to the amino acid sequence of SEQ ID NO: 31. In some embodiments, the N-terminal fragment of a hygromycin resistance protein comprises an amino acid sequence having at least 98% sequence identity to the amino acid sequence of SEQ ID NO: 31. In some embodiments, the N-terminal fragment of a hygromycin resistance protein comprises an amino acid sequence having at least 99% sequence identity to the amino acid sequence of SEQ ID NO: 31. In some embodiments, the N-terminal fragment of a hygromycin resistance protein comprises the amino acid sequence of SEQ ID NO: 31, wherein SEQ ID NO: 31 is:

MKKPELTATSVEKFLIEKFDSVSDLMQLSEGEESRAFSFDVGGRGYVLRV
NSCADGFYKDRYVYRHFASAALPIPEVLDIGEFSESLTY.

In some embodiments, the C-terminal fragment of a hygromycin resistance protein comprises an amino acid sequence having at least 90% sequence identity to the amino acid sequence of SEQ ID NO: 29. In some embodiments, the C-terminal fragment of a hygromycin resistance protein comprises an amino acid sequence having at least 95% sequence identity to the amino acid sequence of SEQ ID NO: 29. In some embodiments, the C-terminal fragment of a hygromycin resistance protein comprises an amino acid sequence having at least 96% sequence identity to the amino acid sequence of SEQ ID NO: 29. In some embodiments, the C-terminal fragment of a hygromycin resistance protein comprises an amino acid sequence having at least 97% sequence identity to the amino acid sequence of SEQ ID NO: 29. In some embodiments, the C-terminal fragment of a hygromycin resistance protein comprises an amino acid sequence having at least 98% sequence identity to the amino acid sequence of SEQ ID NO: 29. In some embodiments, the C-terminal fragment of a hygromycin resistance protein comprises an amino acid sequence having at least 99% sequence identity to the amino acid sequence of SEQ ID NO: 29. In some embodiments, the C-terminal fragment of a hygromycin resistance protein comprises the amino acid sequence of SEQ ID NO: 29, wherein SEQ ID NO: 29 is:

CADGFYKDRYVYRHFASAALPIPEVLDIGEFSESLTYCISRRSQGVTLQD
LPETELPAVLQPVAEAMDAIAAADLSQTSGFGPFGPQGIGQYTTWRDFIC
AIADPHVYHWQTVMDDTVSASVAQALDELMLWAEDCPEVRHLVHADFGSN
NVLTDNGRITAVIDWSEAMFGDSQYEVANIFFWRPWLACMEQQTRYFERR
HPELAGSPRLRAYMLRIGLDQLYQSLVDGNFDDAAWAQGRCDAIVRSGAG
TVGRTQIARRSAAVWTDGCVEVLADSGNRRPSTRPRAKK.

In some embodiments, the C-terminal fragment of a hygromycin resistance protein comprises an amino acid sequence having at least 90% sequence identity to the amino acid sequence of SEQ ID NO: 33. In some embodiments, the C-terminal fragment of a hygromycin resistance protein comprises an amino acid sequence having at least 95% sequence identity to the amino acid sequence of SEQ ID NO: 33. In some embodiments, the C-terminal fragment of a hygromycin resistance protein comprises an amino acid sequence having at least 96% sequence identity to the amino acid sequence of SEQ ID NO: 33. In some embodiments, the C-terminal fragment of a hygromycin resistance protein comprises an amino acid sequence having at least 97% sequence identity to the amino acid sequence of SEQ ID NO: 33. In some embodiments, the C-terminal fragment of a hygromycin resistance protein comprises an amino acid sequence having at least 98% sequence identity to the amino acid sequence of SEQ ID NO: 33. In some embodiments, the C-terminal fragment of a hygromycin resistance protein comprises an amino acid sequence having at least 99% sequence identity to the amino acid sequence of SEQ ID NO: 33. In some embodiments, the C-terminal fragment of a hygromycin resistance protein comprises the amino acid sequence of SEQ ID NO: 33, wherein SEQ ID NO: 33 is:

CISRRSQGVTLQDLPETELPAVLQPVAEAMDAIAAADLSQTSGFGPFGPQ
GIGQYTTWRDFICAIADPHVYHWQTVMDDTVSASVAQALDELMLWAEDCP
EVRHLVHADFGSNNVLTDNGRITAVIDWSEAMFGDSQYEVANIFFWRPWL
ACMEQQTRYFERRHPELAGSPRLRAYMLRIGLDQLYQSLVDGNFDDAAWA
QGRCDAIVRSGAGTVGRTQIARRSAAVWTDGCVEVLADSGNRRPSTRPRA
KK.

### Genes of Interest

The methods and systems of the present disclosure are used, in some embodiments, to produce multi-transgenic (e.g., double and/or triple transgenic) cells and/or organisms. Thus, in some embodiments, one vector in the system described herein comprises a first gene of interest that encodes a first molecule (a first molecule of interest), and another vector comprises a second gene of interest that encodes a second molecule (a second molecule of interest).

In some embodiments, the first molecule is a protein. In some embodiments, the second molecule is a protein. Examples of proteins of interest include, but are not limited to, enzymes, cytokines, transcription factors, and growth factors. In some embodiments, proteins of interest include those known in the art which are related to plant stress tolerance (abiotic stress tolerance), yield, biomass, and/or disease resistance. Some non-limiting examples of proteins that are involved in and/or related to plant stress tolerance include but are not limited to: Dehydration-Responsive Element-Binding Protein (DREB1), C-repeat/DRE Binding Factor (CBF), Sodium/Hydrogen Antiporter (NHX1), HVA1 (Dehydrin), and/or late embryogenesis abundance protein (LEA). Some non-limiting examples of proteins that are involved in and/or related to Yield and Biomass include but are not limited to: Gibberellin Oxidase (AtGA20ox), Isopentenyl transferase (IPT), Growth-Regulating Factor (GRF), Grain Number 1a (Gn1a), and/or Ideal Plant Architecture 1 (OsSPL14). Some non-limiting examples of proteins that are involved in and/or related to disease resistance include but are not limited to Nonexpresser of PR Genes 1 (NPR1), pathogenesis-related (PR) proteins, Rice Bacterial Blight Resistance proteins (Xa21), and/or resistance genes (R-genes e.g., RPS2, RPP5).

In some embodiments, the first molecule is a peptide. In some embodiments, the second molecule is a peptide.

### Plants

In some embodiments, the plant is an herbaceous plant. In some embodiments, the herbaceous plant is selected from the group comprising Nicotiana, Arabidopsis thaliana, Brassica rapa, Glycine max, Nicotiana benthamiana, Oryza sativa, Solanum lycopersicum, Solanum tuberosum, Panicum virgatum, Sorghum bicolor, and Zea mays.

In some embodiments, the plant is a woody plant. In some embodiments, the woody plant is selected from the group comprising Citrus sinensis, Eucalyptus grandis, Malus domestica, Populus tremula x P. alba INRA 717-1B4, Prunus persica, Vitis vinifera.

### Methods of Co-Transforming Plant Cells

Another aspect of the current disclosure is directed to a method of co-transforming plant cells using the split selectable marker system for plant co-transformation as described herein. In some embodiments, the method comprises delivering DNA vectors into a plant cell:
A) a first vector comprising:
   (i) from 5′ to 3′, and operably linked:
      a first promoter,
      a nucleotide sequence encoding an N-terminal fragment of a selectable marker protein;
      a nucleotide sequence encoding an N-terminal fragment of an intein; and
      a first terminator;
      wherein the nucleotide sequence encoding the N-terminal fragment of the selectable marker protein is linked, in frame, to the nucleotide sequence encoding the N-terminal fragment of the intein; and
   (ii) a first gene of interest, wherein the first gene of interest comprises a promoter, a coding sequence, and a terminator;
and
B) a second vector comprising:
   (i) from 5′ to 3′, and operably linked:
      a second promoter,
      a nucleotide sequence encoding a C-terminal fragment of the intein;
      a nucleotide sequence encoding a C-terminal fragment of the selectable marker protein; and
      a second terminator;
      wherein the nucleotide sequence encoding the C-terminal fragment of the intein is linked, in frame, to the nucleotide sequence encoding the C-terminal fragment of the selectable marker protein; and
   (ii) a second gene of interest, wherein the second gene of interest comprises a promoter, a coding sequence, and a terminator;
wherein upon expression in a plant cell, the N-terminal fragment and the C-terminal fragment of the intein join the N-terminal fragment and the C-terminal fragment of the selectable marker protein to form a peptide bond.

In some embodiments, delivery of the vectors of the disclosed system to the targeted plant cells. Methods of transforming plants are known in the art. In some embodiments, the transformation is a stable transformation. As used herein, stable transformation means that the gene will be fully integrated into the host genome and is expressed continuously. The gene in a stable transformation will also be expressed in later generations of the plant. There are numerous proven genetic transformation methods in the art that can stably introduce new genes into the nuclear genomes of different plant species. However, despite decades of technological advancement, efficient plant transformation and regeneration remain a challenge for many species. Exogenous genes can be delivered to plant cells by Agrobacterium, particle bombardment/gene gun, electroporation, the pollen tube pathway, and other known mediated delivery methods.

One method of plant transformation known in the art is Agrobacterium-mediated plant transformation. As a genus, Agrobacterium can transfer DNA to a remarkably broad group of organisms including numerous dicot and monocot angiosperm species and gymnosperms. Additionally, Agrobacterium can transform fungi, yeasts, ascomycetes, and basidiomycetes. This is known as the most common method of plant transformation. The Agrobacterium's DNA is engineered to carry the desired gene, and the Agrobacterium naturally transfers its T-DNA into the plant cells.

A subtype of Agrobacterium transformation is the floral dip method. In this method, transformation of female gametes is accomplished by simply dipping developing Arabidopsis inflorescences for a few seconds into a 5% sucrose solution containing 0.01-0.05% (vol/vol) Silwet L-77 and resuspended Agrobacterium cells carrying the nucleic acids or vectors to be transferred. Treated plants are allowed to set seed which are then plated on a selective medium to screen for transformants. A transformation frequency of at least 1% can be routinely obtained and a minimum of several hundred independent transgenic lines generated from just two pots of infiltrated plants (20-30 plants per pot) within 2-3 months.

Another method of plant transformation is particle bombardment (also known as gene gun). This method involves coating the target gene on the surface of gold or tungsten powder to construct a DNA-coated microcarrier. High-pressure helium pulses accelerate the DNA-coated microcarrier into the gas acceleration tube using an electric discharge or a pressurized helium gas stream. These particles gain sufficient momentum to pierce recipient cells at high speed, while the target gene coated on the outside remains in the cell and is eventually integrated into the plant's chromosome, producing the transformed plant.

Another method of transformation of plant cells is electroporation. Electroporation uses short, high-field electrical pulses to create transient pores in the plasma membrane of target cells, increasing the permeability of the host cell membrane. Under an optimal electrical pulse, these pores can be resealed, restoring the cells to their original state. Compared to Agrobacterium and particle-bombardment-mediated plant transformation, electroporation-mediated transformation has the advantages of rapid application, low cost, and a highly stable transformation rate.

Another known method of transforming plant cells is the pollen tube pathway-mediated transformation. The pollination process of higher plants, pollen forms the pollen tube after germination on the stigma surface and extends to the ovule along the style, and the pollen nucleus passes through the pollen tube to fertilize the ovule. Pollen-tube-mediated plant genetic transformation entails removing the stigma from the recipient plant immediately after pollination and adding exogenous DNA solution dropwise to the recipient plant's severed style. The exogenous DNA is transported to the recipient plant's ovary by pollen tube growth, where it is integrated with the undivided but fertilized recipient egg, resulting in the exogenous DNA being integrated into the recipient's genome at the embryogenic stage and being present in the transformed seed.

Liposome-Mediated Plant Genetic Transformation is also known in the art. Liposomes are spherical vesicles composed of one or more phospholipid bilayer membranes, ranging in size from 30 nm to several micrometers, and composed of cholesterol and natural nontoxic phospholipids. According to the size and number of bilayer membranes, liposomes can be divided into two types: multilamellar vesicles (MLV) and unilamellar vesicles. The latter is further classified into large unilamellar vesicles (LUV) and small unilamellar vesicles (SUV). Liposome-mediated transformation can introduce exogenous DNA into protoplasts through plasma membrane fusion or protoplast endocytosis. Liposomes and DNA are mixed and incubated to form a DNA-lipid complex, which is subsequently mixed with protoplast suspension (supplemented with PEG), and the desired DNA is introduced into the target protoplast through liposome-protoplast fusion or endocytosis. The positively charged liposome is attracted to the negatively charged DNA and the cell membrane, enabling adhesion of the liposome to the protoplast surface, followed by the incorporation of the liposome and protoplast at their binding sites, and finally releasing the plasmid into the target cells.

Silicon-Carbide-Whisker-Mediated Transformation is also known as a method of transforming plant cells. Silicon carbide whiskers (SCWs) consist of needle-like microwhiskers with a diameter of about 0.5 μm and a length of about 10-80 μm. The whiskers are tough and easily cleaved, resulting in sharp cutting edges that pierce the cell wall and eventually the cell nucleus. SCW-mediated plant genetic transformation is achieved by placing suspended cells or embryogenic calli and DNA in a centrifuge tube containing SCW, which cannot bind to DNA due to its negatively charged surface. Through vortexing, SCWs can create needle-like pores on the cell membrane through which exogenous DNA can enter the target cells

In microinjection-mediated plant genetic transformation, DNA is injected into a single plant nucleus or cytoplasm using a glass microcapillary injection pipette. In this technique, the target cell is fixed under a microscope; there are two micromanipulators, one of which is the holding pipette that fixes the cell and the other is a microcapillary tube containing a small amount of DNA solution to penetrate the cell membrane or nuclear membrane. Through injection, the DNA is transferred into the cytoplasm/nucleus of plant cells or protoplasts using the microcapillary pipette (0.5-10 μm at the tip), and the transformed cells are cultured and grown into transgenic plants after gene transfer is completed.

In some embodiments, the vectors (vector pairs) described herein utilize a split selectable marker and a split intein and are utilized in the method for co-transformation of plant cells. As such, the vectors described herein regarding the system of the co-transformation are all applicable to the method of co-transforming plant cells. Therefore, all the various embodiments described above relating to the options of intein, selectable markers, the split sites of the intein and selectable markers, choices of promoters and terminators, are all incorporated into this section for the method.

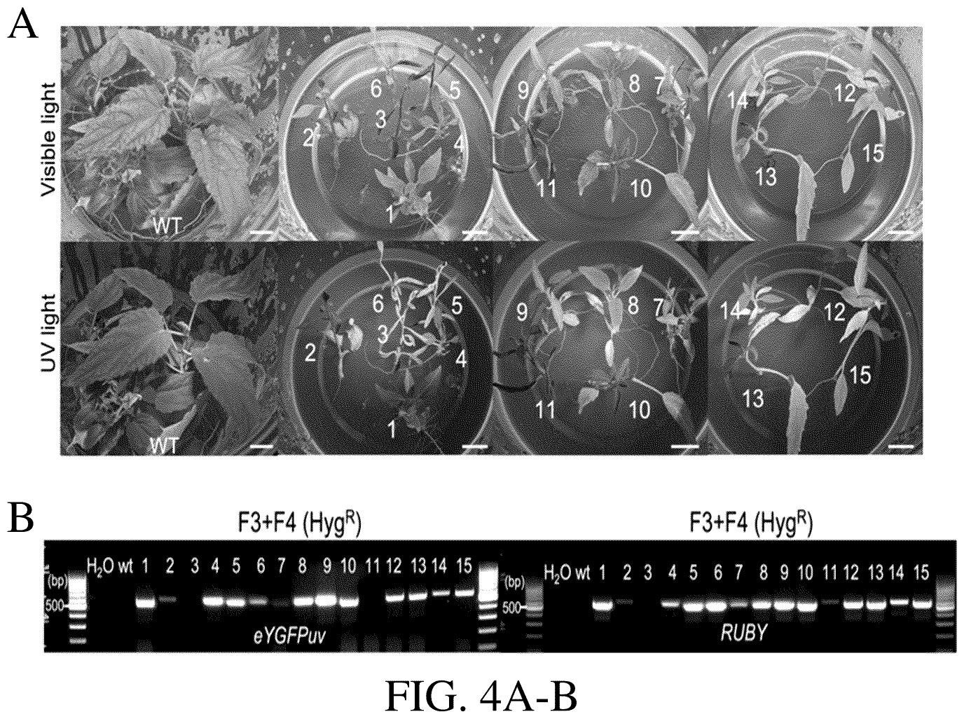

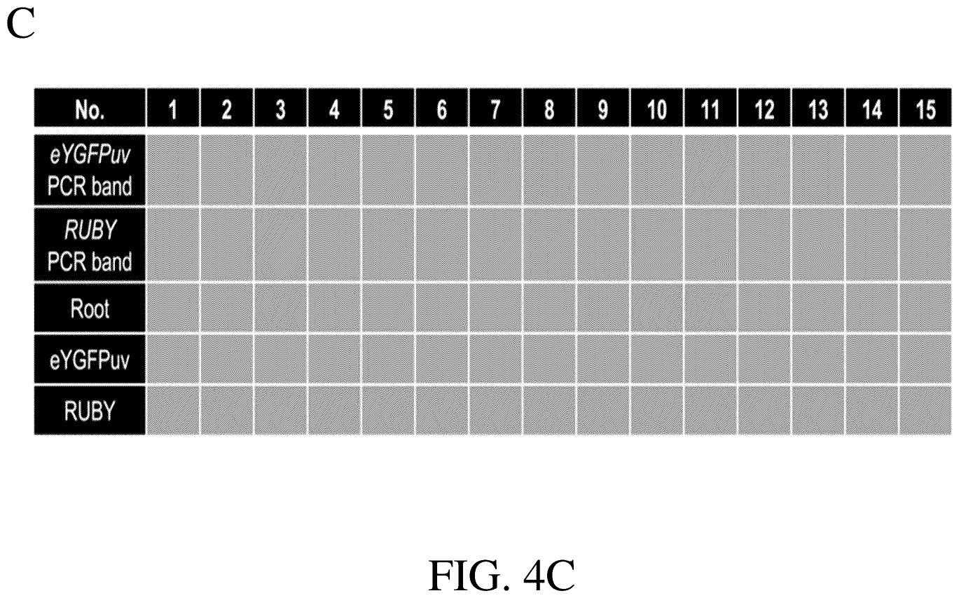

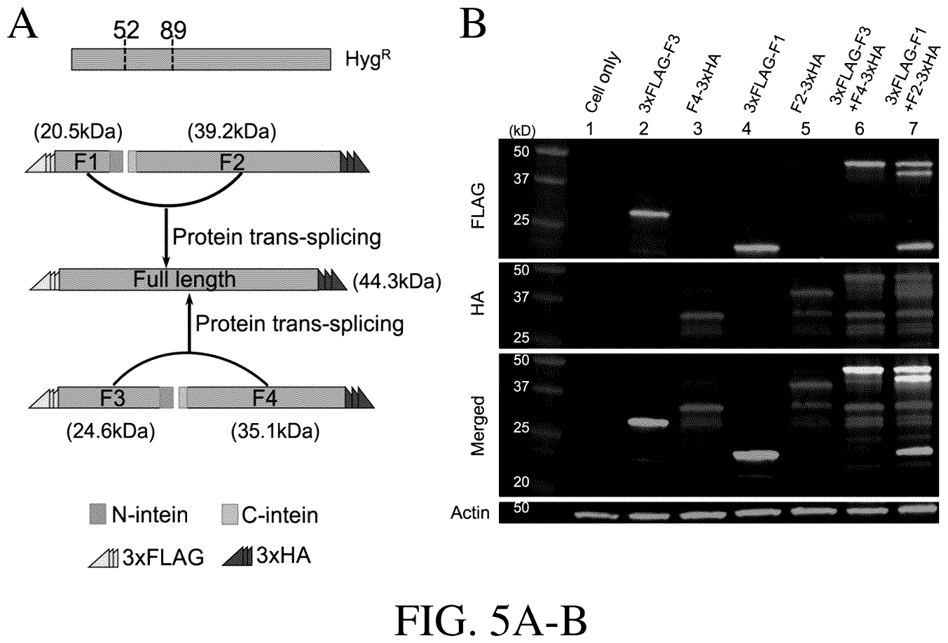

Successful Co-Transformation of the disclosed methods can be seen through methods known in the art. Successful co-transformation can be detected by phenotype. For example, transgenic seedlings with typical antibiotic-resistant phenotype are successfully identified on the selection media comprising the antibiotic. Such a phenotype indicates that the two inactive fragments of the selectable marker gene (antibiotic resistance) were effectively reconstituted post-translationally. Additionally, red pigment can be observed in transformants which receive RUBY or green fluorescence can be seen in plants transformed with eYGFPuv. To check successful co-transformation, the pigment or fluorescence can be seen at different stages of antibiotic-resistant T1 plants. Such phenotypes suggest that both the visual vectors and the resistance vectors were transformed through the split-mediated co-transformation (see FIGS. 3A, 3B, and 4A). In some embodiments, the transformants can be genotyped through methods readily known in the art. Such genotyping includes PCR-based genotyping, which can readily show the presence of the visual markers in plants that are also antibiotic-resistant (see FIG. 4B). Additional methods of confirming co-transformation include western blot analysis of protein trans-splicing between N-terminal peptide fragments of antibody resistance which are N-terminally tagged with 3xFLAG-epitope and C-terminal peptide fragments of antibody resistance C-terminally tagged with 3xHA-epitope. Western blot analysis detecting the FLAG epitopes from protein extractions allows for confirmation that the transformation occurred (see FIG. 5B).

In some embodiments, the co-transformation is at least 60% efficient. In some embodiments, the co-transformation is at least 65% efficient. In some embodiments, the co-transformation is at least 70% efficient. In some embodiments, the co-transformation is at least 65% efficient. In some embodiments, the co-transformation is at least 75% efficient. In some embodiments, the co-transformation is at least 80% efficient. In some embodiments, the co-transformation is at least 85% efficient.

In some embodiments, the intein is NpuDnaE. In some embodiments, the split between the N-terminal fragment and the C-terminal fragment of NpuDnaE occurs at amino acid position within the first 110 amino acids of SEQ ID NO: 9. In some embodiments, the split between the N-terminal fragment and the C-terminal fragment of NpuDnaE occurs at amino acid position N102:I103.

In some embodiments, the first promoter and the second promoter are each an inducible promoter. In some embodiments, the first promoter and the second promoter are each a constitutive promoter. In some embodiments, the first promoter is an inducible promoter, and the second promoter is a constitutive promoter. In some embodiments, the first promoter is a constitutive promoter, and the second promoter is an inducible promoter.

In some embodiments, the selectable marker protein is a protein that produces a visible signal. In some embodiments, the visible signal is a red pigment. In some embodiments, the selectable marker protein is RUBY. In some embodiments, the selectable marker protein is RUBY, and the split between the N-terminal fragment and the C-terminal fragment of RUBY occurs at an amino acid position within the first 240 amino acids of SEQ ID NO: 1. In some embodiments, the selectable marker protein is RUBY, and the split between the N-terminal fragment and the C-terminal fragment of RUBY occurs at amino acid position L231:C232 of SEQ ID NO: 1.

In some embodiment, the visible signal is a fluorescent signal. In some embodiments, the selectable marker protein is eYGFPuv. In some embodiments, the selectable marker protein is eYGFPuv, and the split between the N-terminal fragment and the C-terminal fragment of eYGFPuv occurs at an amino acid position within the first 75 amino acids of SEQ ID NO: 3. In some embodiments, the selectable marker protein is eYGFPuv, and the split between the N-terminal fragment and the C-terminal fragment of eYGFPuv occurs at amino acid position T52:C53 of SEQ ID NO: 3.

In some embodiments, the selected marker protein is a protein encoded by an antibiotic resistance gene. In some embodiments, the antibiotic resistance gene is a kanamycin resistance gene. In some embodiments, the selectable marker protein is a protein encoded by kanamycin resistance gene, and the split between the N-terminal fragment and the C-terminal fragment of the protein occurs at an amino acid position within the first 200 amino acids of SEQ ID NO: 5. In some embodiments, the selectable marker protein is a protein encoded by kanamycin resistance gene, and the split between the N-terminal fragment and the C-terminal fragment of the protein occurs at amino acid position T131:C132 or A192:C193 of SEQ ID NO: 5.

In some embodiments, the antibiotic resistance gene is a hygromycin resistance gene. In some embodiments, the selectable marker protein is a protein encoded by hygromycin resistance gene, and the split between the N-terminal fragment and the C-terminal fragment of the protein occurs at an amino acid position within the first 100 amino acids of SEQ ID NO: 7. In some embodiments, the selectable marker protein is a protein encoded by hygromycin resistance gene, and the split between the N-terminal fragment and the C-terminal fragment of the protein occurs at amino acid position S52:C53 of SEQ ID NO: 7. In some embodiments, the selectable marker protein is a protein encoded by hygromycin resistance gene, and the split between the N-terminal fragment and the C-terminal fragment of the protein occurs at amino acid position Y89:C90 of SEQ ID NO: 7.

In some embodiments, the plant is an herbaceous plant. In some embodiments, the herbaceous plant is selected from the group comprising Nicotiana, Arabidopsis thaliana, Brassica rapa, Glycine max, Nicotiana benthamiana, Oryza sativa, Solanum lycopersicum, Solanum tuberosum, Panicum virgatum, Sorghum bicolor, and Zea mays.

In some embodiments, the plant is a woody plant. In some embodiments, the woody plant is selected from the group comprising Citrus sinensis, Eucalyptus grandis, Malus domestica, Populus tremula x P. alba INRA 717-1B4, Prunus persica, Vitis vinifera.

### EXAMPLES

The following examples are set forth as being representative of the present disclosure. These examples are not to be construed as limiting the scope of the present disclosure as these and other equivalent embodiments will be apparent in view of the present disclosure, figures and accompanying claims.

### Example 1: Intein-Mediated Split RUBY Reporter in Tobacco Leaf Infiltration

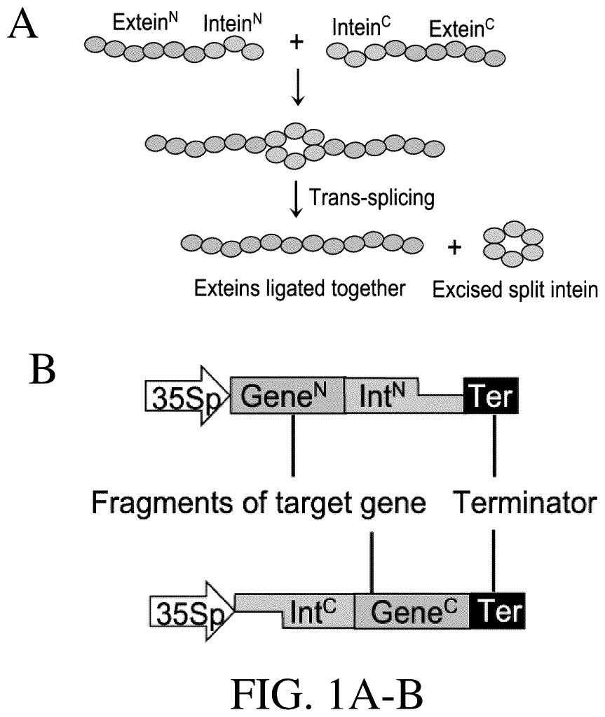

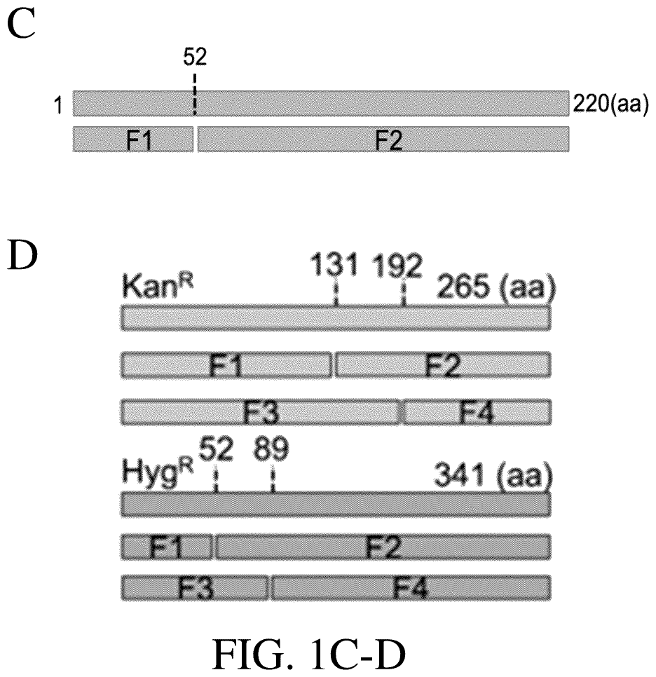

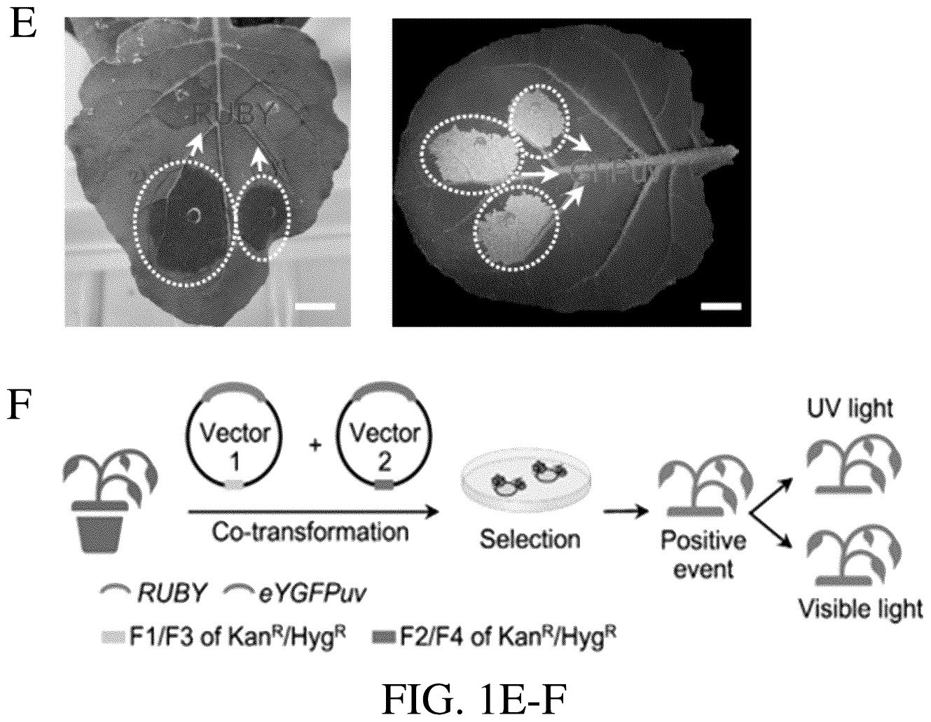

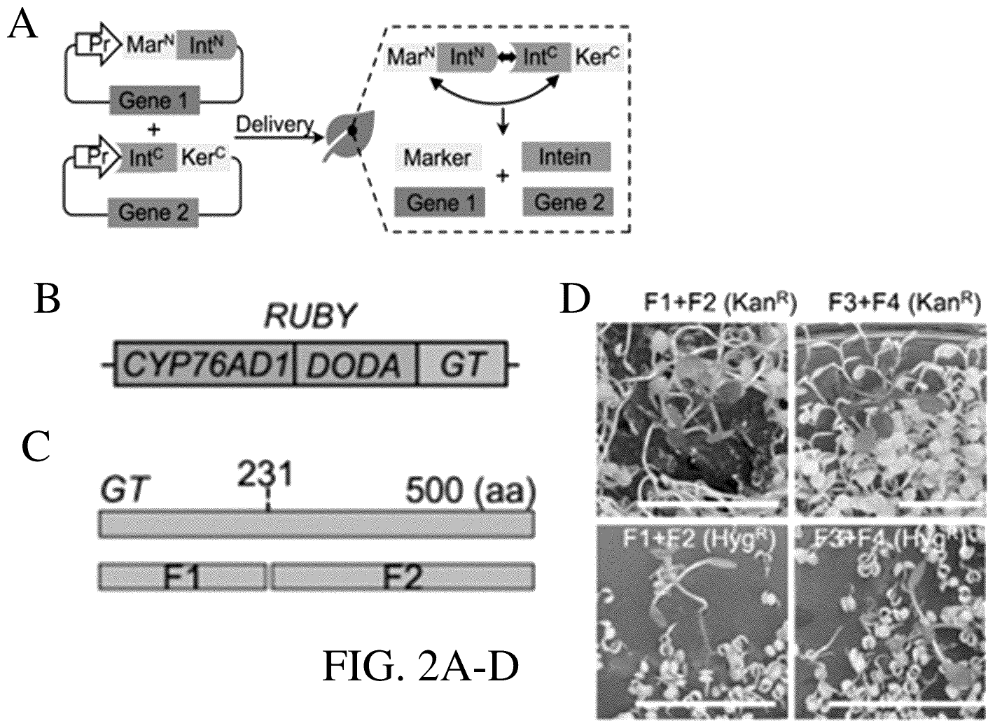

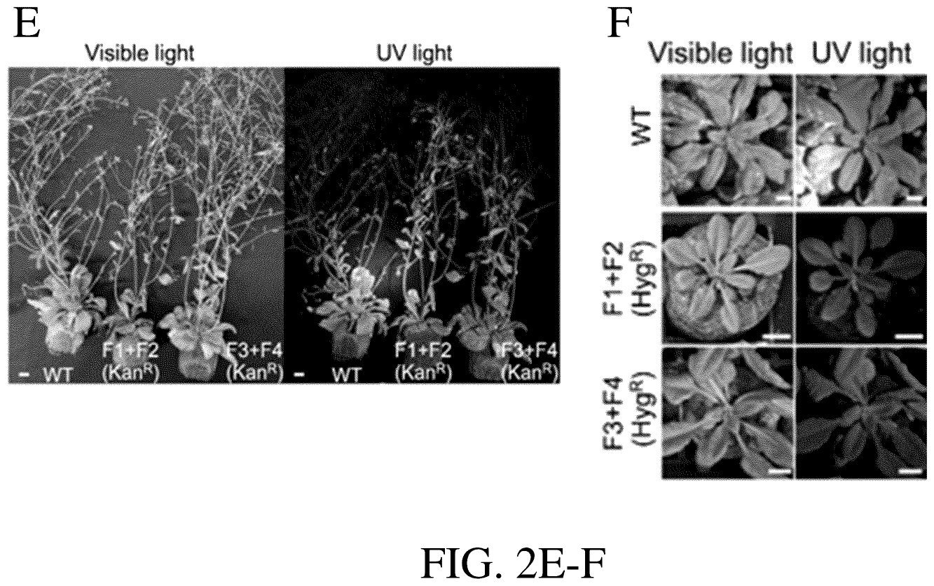

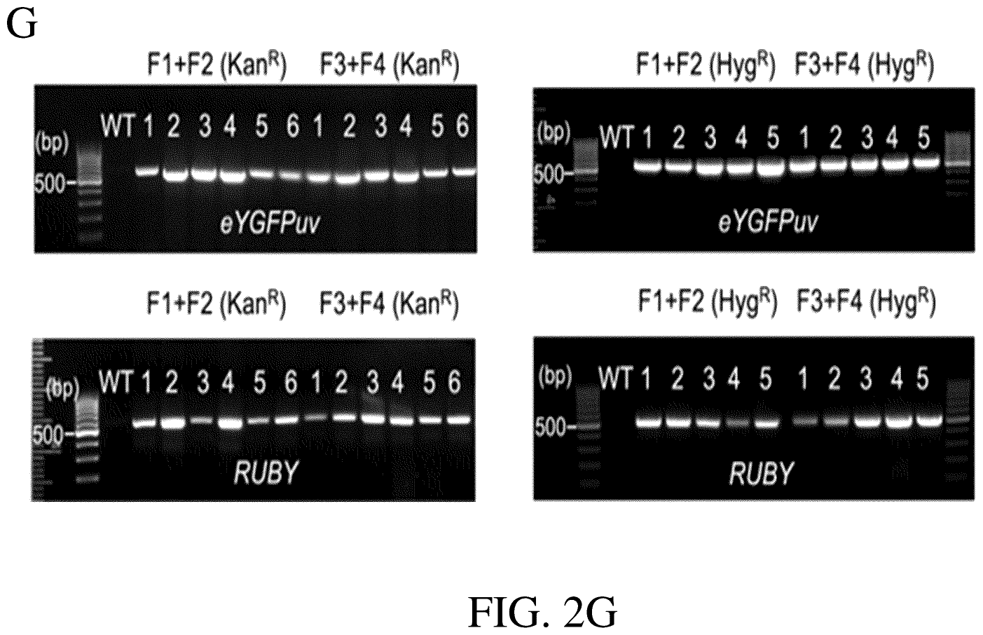

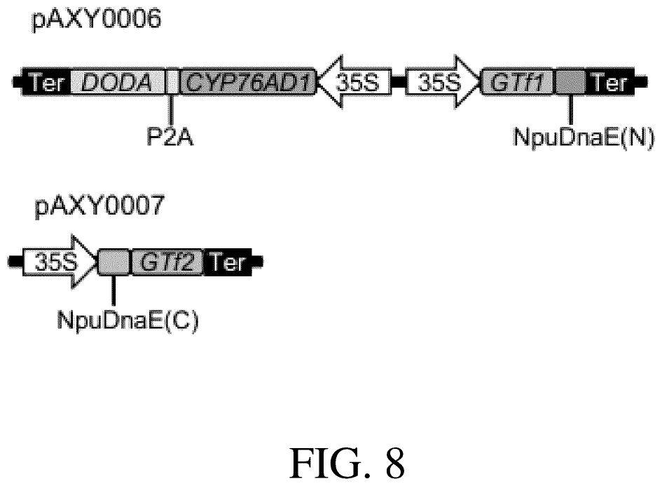

Initially, eYGFPuv11 and RUBY12 were selected as the reporter genes that can be easily visualized by naked eyes with and without UV light, respectively, to establish a functional split system. In fact, the RUBY reporter is encoded by three genes CYP76AD1, DODA, and glucosyltransferase (GT) (FIG. 2B). In general, a catalytic Cys residue at position +1 of the C-terminal extein is mandatory to maintain substantial splicing activities7. A potential split site, L231:C232, was thus identified for splitting the gene GT into two fragments, termed GTf1 and GTf2 (FIG. 2C). The RUBY reporter was split into two parts by creating plasmid pAXY0006 containing CYP76AD1, DODA and GTf1-NpuDnaE (N), and plasmid pAXY0007 containing NpuDnaE (C)-GTf2 (FIG. 8). Note that the Arabidopsis codon-optimized NpuDnaE intein was created to improve gene expression and translational efficiency in plants. Split-RUBY was tested using Agrobacterium-mediated leaf infiltration in Nicotiana benthamiana. The strong red pigment was observed by naked eyes both in the positive control (RUBY) and the leaf area co-infiltrated with pAXY0006 and pAXY0007 plasmids, whereas no red pigment was detected in the leaf area infiltrated with pAXY0006 or pAXY0007 plasmids alone (negative controls) (FIG. 1E). Taken together, these results indicate that the functional RUBY reporter was restored by split inteins, which is consistent with the split eYGFPuv reported previously (Yuan, G. et al. ACS Synth. Biol. 11, 2513-2517 (2022)).

### Example 2: The Intein-Mediated Split Selectable Marker in Arabidopsis

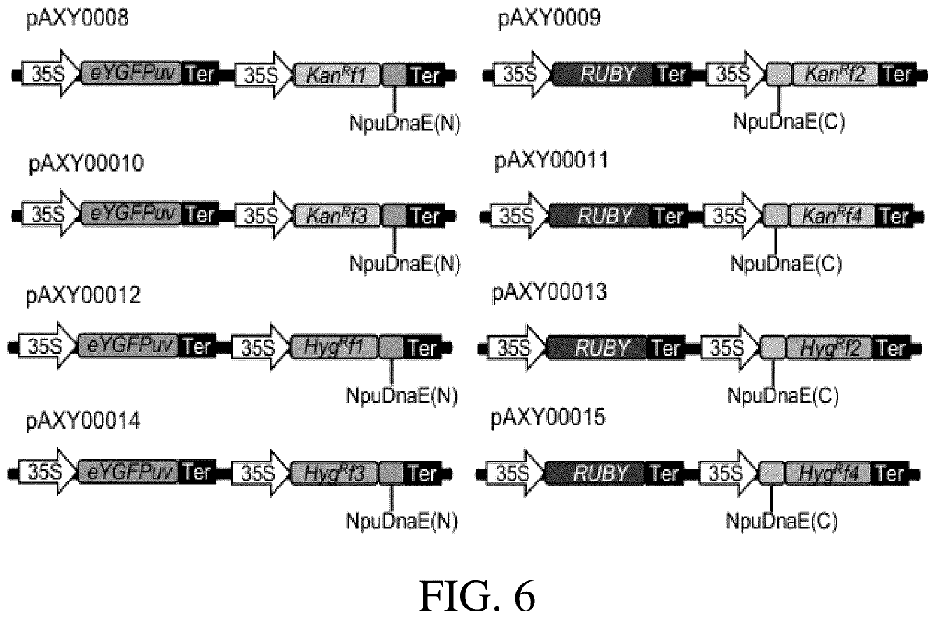

Because Kanamycin resistance (KanR; nptII) and Hygromycin resistance (HygR; hpt) are widely used as the selectable markers in plant transformation, NpuDnaE intein was tested for splitting the nptII gene encoding neomycin phosphotransferase II and the hpt gene encoding Hygromycin phosphotransferase, which confers KanR and HygR, respectively. Following the rule of obligatory cysteine residue on the C-extein, two split sites were identified for nptII (T131:C132 and A192:C193) and two split sites for hpt (S52:C53 and Y89:C90) (FIG. 1D). The coding sequence of nptII or hpt was split into an N-terminal fragment (MarN, F1/F3) and a C-terminal fragment (KerC, F2/F4), which were then cloned upstream of an N-terminal fragment of the NpuDnaE intein (IntN) and downstream of a C-terminal fragment of the NpuDnaE intein (IntC), respectively, into two vectors (FIGS. 1D and 2A). Each vector also carries one of the two reporters (eYGFPuv and RUBY), which allows for easy assessment of co-transformation (FIG. 6). Thus, expectations were to see both green fluorescence under UV light and red pigment under visible light in Kanamycin-resistant or Hygromycin-resistant transgenic plants after co-transformation of these two vectors (FIG. 1F).

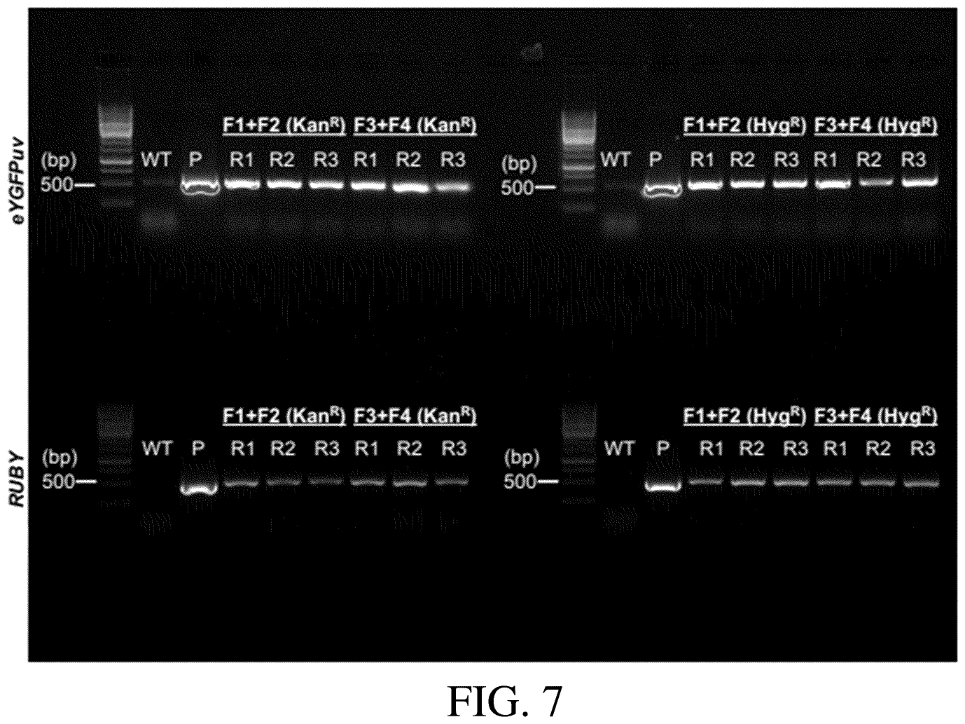

After co-transformation via a floral dip in Arabidopsis, multiple transgenic seedlings with typical Kanamycin-resistant or Hygromycin-resistant phenotype were successfully identified on the selection media, indicating that the two inactive fragments of each selectable marker gene (nptII or hpt) were effectively reconstituted post-translationally (FIG. 2D). Subsequently, the green fluorescence and red pigment were observed at different stages of Kanamycin-resistant T1 plants (FIG. 2E) and Hygromycin-resistant T1 plants (FIG. 2F) under UV light and visible light, respectively, suggesting that both eYGFPuv and RUBY vectors were also transformed into the same plant simultaneously through split-KanR- or split-HygR-mediated co-transformation. These observations were further confirmed by polymerase chain reaction (PCR)-based genotyping, where both eYGFPuv and RUBY genes were detected in all Kanamycin-resistant or Hygromycin-resistant plants (FIG. 2G). Next, the phenotype of T2 generations of above transgenic plants were evaluated, with the expectation that the Kanamycin-resistance or Hygromycin-resistance phenotype will be observed in T2 seedlings, along with green fluorescence under UV light and red pigment under visible light, as seen in FIG. 3A. Due to segregation of T-DNA inserts in the offspring, not all T2 plants exhibit the phenotype of antibiotic resistance and expression of both eYGFPuv and RUBY, but the phenotype of eYGFPuv and RUBY was observed in most resistant T2 plants though the expression level varies among plants (FIG. 3A). The phenotypes of eYGFPuv and RUBY were continuously detected in the mature plants of Kanamycin-resistant lines and Hygromycin-resistant lines (FIG. 3B). These findings were also supported by PCR-based genotyping, which revealed the presence of the eYGFPuv and RUBY genes in every plant that was either kanamycin- or hygromycin-resistant (FIG. 7). Taken together, the traits of Kanamycin resistant, Hygromycin-resistant, eYGFPuv and RUBY are all heritable in Arabidopsis across generations, demonstrating the split-KanR- and split-HygR-mediated co-transformation are effective and robust methods for stable gene-stacking in plants.

### Example 3: The Intein-Mediated Split Selectable Marker in Poplar

The efficacy of split-HygR system in poplar using vector pairs F3 and F4 was then examined. After tissue-culture-based co-transformation in Poplar ‘717’ (Populus tremula x alba clone INRA ‘717-1B4’), more than twenty transgenic shoots that showed bright green fluorescence under UV light were observed. Fifteen eYGFPuv-expressing shoots were randomly selected and cultured on a root induction medium supplied with Hygromycin (FIG. 4A), where 80% of induced shoots were rooted successfully on the selection medium, suggesting that functional Hygromycin phosphotransferase was generated post-translationally. Consistent green fluorescence was observed in all rooted plants over time, though some plants showed weaker and nonuniform eYGFPuv signals (FIG. 4A). Interestingly, typical RUBY phenotype was not observed in the rooted plants though red pigmentation was observed in the stem of some plants. Based on the RUBY expression in transgenic poplar plants generated previously, red pigment is typical to be visible in different organs, including leaf, stem, and root (Yuan, G. et al. Hortic. Res. https://doi.org/10.1093/hr/uhac077 (2022)). However, the red pigmentation in the stem of some plants can also be caused by stress, which is not rare during plant transformation. Intriguingly, both eYGFPuv and RUBY genes were detected via PCR genotyping in all rooted plants (FIGS. 4B and 4C). Overall, these results suggest that the split-HygR system can also work effectively in tissue-culture-based transformation in woody plants.

### Example 4: Western Blot Analysis of Protein Trans-Splicing of Hygromycin Markertrons

To directly observe protein splicing and to confirm these inteins are indeed orthogonal, western blot analysis was conducted of protein trans-splicing between N-HygR N-terminally tagged with 3xFLAG-epitope and C-HygR C-terminally tagged with 3xHA-epitope (FIGS. 5A and 8). As expected, full length HygR (lanes 6 and 7) was observed in the co-transformation of cognate HygR fragments with matching N- and C-inteins while transformation with fragment F1/F2/F3/F4 only (lanes 2-5) did not yield full-length HygR (FIG. 5B).

### Example 5: General Methods

### Plant Materials

Arabidopsis (Arabidopsis thaliana) ecotype Columbia-0 (Col-0) and tobacco (Nicotiana benthamiana) were grown in controlled-climate chambers under fluorescent cold white light (100 to 150 μmol m⁻² s⁻¹), 16-h light/8-h dark photoperiod, 20-22° C., and 60% humidity. In vitro-grown poplar ‘717’ (Populus tremula x P. alba clone INRA 717-1B4) plantlets were placed in a growth room with photoperiod of 16-h light/8-h dark at 22° C.

### Vector Construction

To split RUBY, a RUBY-minus vector without the gene GT was first created by assembling PCR product 1 containing CYP76AD1 and DODA, and PCR product 2 containing Arabidopsis HSP18.2 terminator into a pGFPGUSplus vector 17 via NEBuilder HiFi DNA Assembly (New England BioLabs). The pAXY0006 vector of split-RUBY was generated by assembling PCR products containing f1 fragment of gene GT (named GTf1) and NpuDnaE (N) into RUBY-minus vector via NEBuilder HiFi DNA Assembly. The pAXY0007 vector of split-RUBY was generated by assembling PCR products containing f2 fragment of gene GT (named GTf2) and NpuDnaE (C) into pGFPGUSplus vector via NEBuilder HiFi DNA Assembly. To split KanR (i.e., nptII) and HygR (i.e., hpt), gBlocks Gene Fragments containing either 5′-KanR/HygR and N-terminal of NpuDnaE or C-terminal of NpuDnaE and 3′-KanR/HygR were synthesized from Integrated DNA Technologies IDT. The pAXY0008/00010/00012/00014 vectors of split-KanR/HygR were generated by assembling PCR products containing F1/F3 fragment of KanR/HygR and NpuDnaE(N) into pGFPGUSplus vector via NEBuilder HiFi DNA Assembly. The pAXY0009/00011/00013/00015 vectors of split-KanR/HygR were generated by assembling PCR products containing F2/F4 fragment of KanR/HygR and NpuDnaE (C) into pGFPGUSplus vector via NEBuilder HiFi DNA Assembly. The coding sequences of inteins were codon optimized for Arabidopsis via the online codon optimization tool (ExpOptimizer) provided by NovoPro Bioscience (Shanghai, China). All vectors were verified by Sanger sequencing. Information for all primers, gBlocks and plasmids used in this study is provided in Tables 1 and 2.

TABLE 1

| Plasmid # | pXYA# | Plasmid Name |
|---|---|---|
| 1 | pAXY0006 | pGlucos(1-231)-NpuDnaE(N) |
| 2 | pAXY0007 | pNpuDnaE(C)-Glucos (232-500) |
| 3 | pAXY0008 | pKan^{R}(1-131)-NpuDnaE(N)_eYGFPuv. |
| 4 | pAXY0009 | pNpuDnaE(C)_Kan^{R} (132-265)_RUBY. |
| 5 | pAXY0010 | pKan^{R}(1-192)-NpuDnaE(N)_eYGFPuv. |
| 6 | pAXY0011 | pNpuDnaE(C)_Kan^{R} (193-265)_RUBY. |
| 7 | pAXY0012 | pHyg^{R}(1-52)-NpuDnaE(N)_eYGFPuv |
| 8 | pAXY0013 | pNpuDnaE(C)_Hyg^{R} (53-341)_RUBY |
| 9 | pAXY0014 | pHyg^{R}(1-89)-NpuDnaE(N)_eYGFPuv |
| 10 | pAXY0015 | pNpuDnaE(C)_Hyg^{R} (90-341)_RUBY |
| 11 | pAXY0016 | p3flag_Hyg^{R}(1-89)-NpuDnaE(N) |
| 12 | pAXY0017 | pNpuDnaE(C)_Hyg^{R} (90-341)_3HA |
| 13 | pAXY0018 | p3flag_Hyg^{R}(1-52)-NpuDnaE(N) |
| 14 | pAXY0019 | pNpuDnaE(C)_Hyg^{R} (53-341)_3HA |

### Arabidopsis Stable Transformation

The Agrobacterium tumefaciens strain ‘GV3101’ was used for the transformation of Arabidopsis wild type ‘Col-0’ via the floral dip method as described by Yuan et al. For co-transformation, two Agrobacterium strains containing corresponding vectors (FIG. 6), respectively, were cultured separately overnight in 100 mL LB liquid medium supplied with 50 mg/L Kanamycin and 50 mg/L Rifampicin. Two LB cultures were spun down at 4000-5000 rpm for 20 mins and resuspended in 30 mL new LB liquid medium without antibiotics and mix equally. Mixed LB culture was added into 120 mL dip solution containing 5% sucrose and 0.03% Silwet-L77. In general, 8 to 12 plants were used for each co-transformation.

### Poplar Stable Transformation

The Agrobacterium tumefaciens strain ‘EHA105’ was used for the co-transformation of the poplar ‘717’ following a published method (Yuan, G., Tuskan, G. A. & Yang, X. Methods Mol Biol (2022)). 50 mL LB culture for each Agrobacterium strain was prepared and spun down as described above. Two Agrobacterium pellets were resuspended equally in MS induction medium containing 20 μM acetosyringone at an OD600 nm of 0.5-0.8 for each strain. Excised leaf disks from young leaves (˜150) were soaked in Agrobacterium solution for 1 hour and followed by multiple steps including co-culture, washing, callus induction, shoot induction, shoot elongation, and root induction.

### Tobacco Leaf Infiltration

Infiltration of tobacco leaf was performed following a published method (Yuan, G., Tuskan, G. A. & Yang, X. Methods Mol Biol (2022)). For co-infiltration, 5 mL overnight culture of two Agrobacterium strains were spun down and resuspended equally in resuspension solution containing 10 mM MgCl2, 10 mM MES-K (pH 5.6), and 100 μM acetosyringone at an OD600 nm of 0.5 for each strain.

### Genotyping

To genotype the resistant lines, leaves, approximately 0.5-1.0 cm, were collected from Arabidopsis and poplar ‘717’, and ground well. Genomic DNA was isolated by a modified sodium dodecyl sulfate (SDS) based DNA extraction method. Forward primer 5′-CACGGCAACCTCAACG-3′ (SEQ ID NO: 39) and reverse primer 5′-CTCGACACGTCTGTGGG-3′ (SEQ ID NO: 40) were used for genotyping PCR of eYGFPuv. Forward primer 5′-CAGAGCTTGCGAGAAAGG-3′ (SEQ ID NO: 41) and reverse primer 5′-GGCGGAGGTGAACTTGTAG-3′ (SEQ ID NO: 42) were used for genotyping PCR of RUBY.

### Phenotyping

The fluorescence signals of eYGFPuv were visualized under a 365 nm wave-length UV light and imaged using an iphone 11 as described by Yuan et al. The red pigment due to RUBY expression is visible by naked eyes without requiring any equipment and images were also taken using an iPhone 11.

### Protein Extraction and Western Blot

HEK 293T cells were obtained from ATCC and maintained in a humidified atmosphere at 5% CO2 in Dulbecco's Modified Eagle's (DMEM) complete medium (Corning) supplemented with 10% fetal bovine serum (FBS; Seradigm) in 37° C. Plasmid transfections were done with TransIT-LT1 (Mirus Bio) per the manufacturer's instructions. Briefly, cell extracts were generated on ice in EBC buffer, 50 mM Tris (pH 8.0), 120 mM NaCl, 0.5% NP40, 1 mM DTT, and protease and phosphatase inhibitors tablets (Thermo Fisher Scientific). Extracted proteins were quantified using the Pierce™ BCA Protein assay kit (Thermo Fisher). Proteins were separated by SDS acrylamide gel electrophoresis and transferred to IMMOBILON-FL 26 PVDF membrane (Millipore) probed with the indicated antibodies and visualized either by chemiluminescence (according to the manufacturer's instructions) or using a LiCor Odyssey infrared imaging system.

TABLE 2

| | | SEQ | |
|---|---|---|---|
| Name | Sequence | ID NO: | Usage |
| 608_HSPT87 | GACTACCAAAAAATTCTAAGAAACAG | 43 | Cloning primer for |
| | | | pAXY0007 vector |
| 624_cloningF | TCATTTGGAGAGAACACGGGGGACTCTAGA | 44 | Cloning primer for |
| | ATGGATCATGCGACCCT | | RUBY vector without |
| | | | gene glucosyltrans- |
| | | | ferase (GT) |
| 625_cloningR | AGGAAACAGCTATGACATGATTACGAATTC | 45 | Cloning primer for |
| | GGGGAAATTCGAGCTGG | | RUBY vector without |
| | | | gene glucosyltrans- |
| | | | ferase (GT) |
| 626_cloningF | TAGATGCCACCTGACGTC | 46 | Cloning primer for |
| | | | vectors pAXY0008, |
| | | | pAXY00010, |
| | | | pAXY00012, |
| | | | pAXY00014 |
| 627_cloningR | ATCTGCGAAAGCTCGAGA | 47 | Cloning primer for |
| | | | vectors pAXY0008, |
| | | | pAXY00010, |
| | | | pAXY00012, |
| | | | pAXY00014 |
| 643_cloningR | GGCGGAGGTGAACTTGTAG | 48 | Cloning primer for |
| | | | RUBY vector without |
| | | | gene glucosyltrans- |
| | | | ferase (GT) |
| 644_cloningF | CGGCTCCTACAAGTTCACCTCCGCCTGATA | 49 | Cloning primer for |
| | GTGAATATGAAGATGAAG | | RUBY vector without |
| | | | gene glucosyltrans- |
| | | | ferase (GT) |
| 645_cloningF | TAATCTGGGGACCTGCAGG | 50 | Cloning primer for |
| | | | pAXY0006 vector |
| 646_cloningR | GTCAAGAGTCCCCCGTG | 51 | Cloning primer for |
| | | | pAXY0006 vector |
| 647_cloningF | GGAGAGAACACGGGGGACTCTTGACATGAC | 52 | Cloning primer for |
| | CGCCATCAAGATG | | pAXY0006 vector |
| 649_cloningF | TGCTTGTCCTACGAGACCGA | 53 | Cloning primer for |
| | | | pAXY0006 vector |
| 650_cloningR | GCTTTCCTGGGCAACGCTTGTTCCACGCGT | 54 | Cloning primer for |
| | TGTGTACAGATATATGTTGAATTATTG | | pAXY0006 vector |
| 651_cloningR | GAATCTCGGTCTCGTAGGACAAGCAGAGCC | 55 | Cloning primer for |
| | AGCCAAAAGACTTC | | pAXY0006 vector |
| 654_cloningF | GCTCTGAAAAATGGATTCATAGCTTCTAAT | 56 | Cloning primer for |
| | TGCAACTCCGTGGAAGAG | | pAXY0007 vector |
| 656_cloningR | CACACCAAATATTTCATCTTCATCTTC | 57 | Cloning primer for |
| | | | pAXY0007 vector |
| 657_cloningF | GATATGAAGATGAAGATGAAATATTTGGTG | 58 | Cloning primer for |
| | | | pAXY0007 vector |
| GB104 | CTATCTCTCTCGAGCTTTCGCAGATATGGG | 59 | gBlock for vectors |
| | GATTGAACAAGATGGATTGCACGCAGGTTC | | pAXY0008, pAXY00010, |
| | TCCGGCCGCTTGGGTGGAGAGGCTATTCGG | | pAXY00012, pAXY00014 |
| | CTATGACTGGGCACAACAGACAATCGGCTG | | |
| | CTCTGATGCCGCCGTGTTCCGGCTGTCAGC | | |
| | GCAGGGGCGCCCGGTTCTTTTTGTCAAGAC | | |
| | CGACCTGTCCGGTGCCCTGAATGAACTCCA | | |
| | GGACGAGGCAGCGCGGCTATCGTGGCTGGC | | |
| | CACGACGGGCGTTCCTTGCGCAGCTGTGCT | | |
| | CGACGTTGTCACTGAAGCGGGAAGGGACTG | | |
| | GCTGCTATTGGGCGAAGTGCCGGGGCAGGA | | |
| | TCTCCTGTCATCTCACCTTGCTCCTGCCGA | | |
| | GAAAGTATCCATCATGGCTGATGCAATGCG | | |
| | GCGGCTGCATACGCTTGATCCGGCTACCTG | | |
| | CTTGTCCTACGAGACCGAGATTCTTACTGT | | |
| | TGAGTATGGATTGCTTCCAATCGGCAAGAT | | |
| | CGTAGAGAAACGTATTGAGTGTACAGTTTA | | |
| | TTCTGTTGACAACAACGGAAATATCTATAC | | |
| | TCAGCCTGTTGCTCAATGGCACGATAGAGG | | |
| | GGAACAAGAGGTTTTCGAGTATTGCCTTGA | | |
| | GGATGGTAGCCTCATTCGAGCAACAAAAGA | | |
| | TCACAAATTTATGACAGTGGATGGTCAGAT | | |
| | GTTACCTATCGATGAAATTTTCGAAAGGGA | | |
| | ATTAGACCTCATGAGAGTGGATAACCTCCC | | |
| | TAATTAGGCTCGAGTTTCTCCATAATAATG | | |
| | TGTGAGTA | | |
| GB105 | GTCTCTCTCTACAAATCTATCTCTCTCGAG | 60 | gBlock for vectors |
| | ATGATTAAGATTGCGACCCGTAAGTACCTG | | pAXY0009, pAXY00011, |
| | GGGAAGCAAAACGTTTACGATATCGGTGTG | | pAXY00013, pAXY00015 |
| | GAGAGAGATCACAATTTTGCTCTGAAAAAT | | |
| | GGATTCATAGCTTCTAATTGCCCATTCGAC | | |
| | CACCAAGCGAAACATCGCATCGAGCGAGCA | | |
| | CGTACTCGGATGGAAGCCGGTCTTGTCGAT | | |
| | CAGGATGATCTGGACGAAGAGCATCAGGGG | | |
| | CTCGCGCCAGCCGAACTGTTCGCCAGGCTC | | |
| | AAGGCGCGCATGCCCGACGGCGAGGATCTC | | |
| | GTCGTGACACATGGCGATGCCTGCTTGCCG | | |
| | AATATCATGGTGGAAAATGGCCGCTTTTCT | | |
| | GGATTCATCGACTGTGGCCGGCTGGGTGTG | | |
| | GCGGACCGCTATCAGGACATAGCGTTGGCT | | |
| | ACCCGTGATATTGCTGAAGAGCTTGGCGGC | | |
| | GAATGGGCTGACCGCTTCCTCGTGCTTTAC | | |
| | GGTATCGCCGCTCCCGATTCGCAGCGCATC | | |
| | GCCTTCTATCGCCTTCTTGACGAGTTCTTC | | |
| | TGAAGTAGATGCCGACCGGATCTGTCGATC | | |
| | GACAAGCTCGAGTTTCTCCATAATAATGTG | | |
| | TGAGTA | | |
| GB118 | ACTTTTAATCAAATCAAGATTAAAGTTAAT | 61 | gBlock for vectors |
| | TAAATGATTAAGATTGCGACCCGTAAGTAC | | pAXY0007 |
| | CTGGGGAAGCAAAACGTTTACGATATCGGT | | |
| | GTGGAGAGAGATCACAATTTTGCTCTGAAA | | |
| | AATGGATTCATAGCTTCTAAT | | |
| GB187 | CATTCTGCCTGGGGACGTCGGAGCAAGCTT | 62 | gBlock for vectors |
| | GATTTAGGTGACACTATAGAATACAAGCTA | | pAXY00016 |
| | CTTGTTCTTTTTGCAGGATCCACCATGGAT | | |
| | TATAAGGATGACGATGACAAAGCAGACTAC | | |
| | AAAGACGACGATGATAAGGCTGATTATAAA | | |
| | GATGATGACGACAAAGGCCGGCCAATGAAG | | |
| | AAACCGGAACTGACTGCAACATCTGTCGAG | | |
| | AAGTTTCTGATTGAAAAGTTCGATTCTGTT | | |
| | TCCGATCTCATGCAGCTCAGTGAGGGCGAA | | |
| | GAGTCCAGAGCTTTCTCATTCGACGTGGGC | | |
| | GGGCGCGGATACGTACTGCGAGTTAATTCT | | |
| | TGTGCCGACGGTTTCTACAAAGATCGGTAT | | |
| | GTCTATCGGCATTTCGCATCTGCCGCACTG | | |
| | CCTATCCCGGAGGTGCTGGACATAGGTGAA | | |
| | TTTAGTGAATCACTGACTTATTGCCTGAGT | | |
| | TATGAGACTGAGATTCTGACCGTAGAATAT | | |
| | GGTCTGCTTCCAATCGGCAAGATTGTGGAA | | |
| | AAAAGGATCGAGTGTACCGTGTATTCTGTG | | |
| | GACAATAACGGCAATATTTACACCCAACCT | | |
| | GTGGCACAGTGGCATGACCGGGGGGAGCAG | | |
| | GAAGTTTTTGAATACTGCTTGGAGGATGGA | | |
| | TCCCTCATTAGAGCTACAAAAGATCACAAG | | |
| | TTTATGACCGTAGACGGACAGATGTTGCCA | | |
| | ATCGACGAGATCTTTGAGAGGGAGCTGGAT | | |
| | CTGATGCGGGTGGACAATCTGCCCAACTAG | | |
| | TCTAGAACTATAGTGAGTCGTATTACGTAG | | |
| GB188 | CATTCTGCCTGGGGACGTCGGAGCAAGCTT | 63 | gBlock for vectors |
| | GATTTAGGTGACACTATAGAATACAAGCTA | | pAXY00018 |
| | CTTGTTCTTTTTGCAGGATCCACCATGGAT | | |
| | TATAAGGATGACGATGACAAAGCAGACTAC | | |
| | AAAGACGACGATGATAAGGCTGATTATAAA | | |
| | GATGATGACGACAAAGGCCGGCCAATGAAG | | |
| | AAACCGGAACTGACTGCAACATCTGTCGAG | | |
| | AAGTTTCTGATTGAAAAGTTCGATTCTGTT | | |
| | TCCGATCTCATGCAGCTCAGTGAGGGCGAA | | |
| | GAGTCCAGAGCTTTCTCATTCGACGTGGGC | | |
| | GGGCGCGGATACGTACTGCGAGTTAATTCT | | |
| | TGCCTGAGTTATGAGACTGAGATTCTGACC | | |
| | GTAGAATATGGTCTGCTTCCAATCGGCAAG | | |
| | ATTGTGGAAAAAAGGATCGAGTGTACCGTG | | |
| | TATTCTGTGGACAATAACGGCAATATTTAC | | |
| | ACCCAACCTGTGGCACAGTGGCATGACCGG | | |
| | GGGGAGCAGGAAGTTTTTGAATACTGCTTG | | |
| | GAGGATGGATCCCTCATTAGAGCTACAAAA | | |
| | GATCACAAGTTTATGACCGTAGACGGACAG | | |
| | ATGTTGCCAATCGACGAGATCTTTGAGAGG | | |
| | GAGCTGGATCTGATGCGGGTGGACAATCTG | | |
| | CCCAACTAGTCTAGAACTATAGTGAGTCGT | | |
| | ATTACGTAG | | |
| GB189 | CATTCTGCCTGGGGACGTCGGAGCAAGCTT | 64 | gBlock for vectors |
| | GATTTAGGTGACACTATAGAATACAAGCTA | | pAXY00017 |
| | CTTGTTCTTTTTGCAGGATCCACCATGATC | | |
| | AAGATCGCCACCCGGAAATACCTCGGCAAA | | |
| | CAGAACGTGTATGATATCGGGGTGGAGAGG | | |
| | GATCATAACTTCGCCCTCAAAAATGGGTTT | | |
| | ATTGCCTCTAATTGCATCTCCCGCAGAAGC | | |
| | CAAGGCGTGACTCTTCAGGACCTTCCAGAG | | |
| | ACTGAACTCCCCGCCGTCCTGCAGCCTGTT | | |
| | GCTGAGGCCATGGACGCCATAGCCGCCGCC | | |
| | GACCTGAGCCAGACCTCTGGTTTTGGGCCT | | |
| | TTCGGCCCTCAGGGAATTGGCCAGTACACC | | |
| | ACCTGGAGGGACTTTATTTGCGCCATCGCC | | |
| | GACCCCCATGTGTATCACTGGCAGACCGTT | | |
| | ATGGACGATACCGTTTCCGCAAGCGTGGCC | | |
| | CAAGCTCTGGATGAGCTGATGCTGTGGGCG | | |
| | GAGGATTGTCCCGAAGTCAGGCATCTTGTC | | |
| | CACGCCGACTTTGGATCTAACAATGTCCTG | | |
| | ACTGATAACGGCAGAATCACTGCCGTAATC | | |
| | GATTGGTCAGAGGCCATGTTCGGCGACTCT | | |
| | CAGTACGAGGTGGCTAACATCTTCTTTTGG | | |
| | AGACCGTGGTTGGCCTGTATGGAACAGCAA | | |
| | ACAAGATACTTCGAGCGCCGGCACCCGGAA | | |
| | CTGGCCGGATCTCCGAGGCTGAGGGCCTAC | | |
| | ATGCTGAGGATAGGACTGGACCAGCTCTAT | | |
| | CAGTCCCTGGTGGATGGAAATTTCGACGAT | | |
| | GCGGCTTGGGCCCAGGGCCGGTGCGACGCA | | |
| | ATAGTGCGGAGCGGAGCTGGGACAGTGGGC | | |
| | CGGACCCAGATTGCCCGGCGATCTGCAGCT | | |
| | GTGTGGACTGATGGCTGTGTTGAGGTGCTG | | |
| | GCTGACAGCGGCAATCGGAGGCCCAGTACG | | |
| | CGACCTAGAGCAAAAAAATACCCATACGAT | | |
| | GTTCCAGATTACGCTTATCCTTATGACGTA | | |
| | CCTGACTATGCATACCCTTATGATGTACCA | | |
| | GACTACGCTTAGTCTAGAACTATAGTGAGT | | |
| | CGTATTACGTAG | | |
| GB190 | CATTCTGCCTGGGGACGTCGGAGCAAGCTT | 65 | gBlock for vectors |
| | GATTTAGGTGACACTATAGAATACAAGCTA | | pAXY00019 |
| | CTTGTTCTTTTTGCAGGATCCACCATGATC | | |
| | AAGATCGCCACCCGGAAATACCTCGGCAAA | | |
| | CAGAACGTGTATGATATCGGGGTGGAGAGG | | |
| | GATCATAACTTCGCCCTCAAAAATGGGTTT | | |
| | ATTGCCTCTAATTGTGCCGACGGTTTCTAC | | |
| | AAAGATCGGTATGTCTATCGGCATTTCGCA | | |
| | TCTGCCGCACTGCCTATCCCGGAGGTGCTG | | |
| | GACATAGGTGAATTTAGTGAATCACTGACT | | |
| | TATTGCATCTCCCGCAGAAGCCAAGGCGTG | | |
| | ACTCTTCAGGACCTTCCAGAGACTGAACTC | | |
| | CCCGCCGTCCTGCAGCCTGTTGCTGAGGCC | | |
| | ATGGACGCCATAGCCGCCGCCGACCTGAGC | | |
| | CAGACCTCTGGTTTTGGGCCTTTCGGCCCT | | |
| | CAGGGAATTGGCCAGTACACCACCTGGAGG | | |
| | GACTTTATTTGCGCCATCGCCGACCCCCAT | | |
| | GTGTATCACTGGCAGACCGTTATGGACGAT | | |
| | ACCGTTTCCGCAAGCGTGGCCCAAGCTCTG | | |
| | GATGAGCTGATGCTGTGGGCGGAGGATTGT | | |
| | CCCGAAGTCAGGCATCTTGTCCACGCCGAC | | |
| | TTTGGATCTAACAATGTCCTGACTGATAAC | | |
| | GGCAGAATCACTGCCGTAATCGATTGGTCA | | |
| | GAGGCCATGTTCGGCGACTCTCAGTACGAG | | |
| | GTGGCTAACATCTTCTTTTGGAGACCGTGG | | |
| | TTGGCCTGTATGGAACAGCAAACAAGATAC | | |
| | TTCGAGCGCCGGCACCCGGAACTGGCCGGA | | |
| | TCTCCGAGGCTGAGGGCCTACATGCTGAGG | | |
| | ATAGGACTGGACCAGCTCTATCAGTCCCTG | | |
| | GTGGATGGAAATTTCGACGATGCGGCTTGG | | |
| | GCCCAGGGCCGGTGCGACGCAATAGTGCGG | | |
| | AGCGGAGCTGGGACAGTGGGCCGGACCCAG | | |
| | ATTGCCCGGCGATCTGCAGCTGTGTGGACT | | |
| | GATGGCTGTGTTGAGGTGCTGGCTGACAGC | | |
| | GGCAATCGGAGGCCCAGTACGCGACCTAGA | | |
| | GCAAAAAAATACCCATACGATGTTCCAGAT | | |
| | TACGCTTATCCTTATGACGTACCTGACTAT | | |
| | GCATACCCTTATGATGTACCAGACTACGCT | | |
| | TAGTCTAGAACTATAGTGAGTCGTATTACG | | |
| | TAG | | |

What is claimed is:

1. A split selectable marker system for plant co-transformation, the system comprising:
A) a first vector comprising:
(i) from 5′ to 3′, and operably linked:
a first promoter,
a nucleotide sequence encoding an N-terminal fragment of a selectable marker protein;
a nucleotide sequence encoding an N-terminal fragment of an intein; and
a first terminator;
wherein the nucleotide sequence encoding the N-terminal fragment of the selectable marker protein is linked, in frame, to the nucleotide sequence encoding the N-terminal fragment of the intein;
and
(ii) a first gene of interest, wherein the first gene of interest comprises a promoter, a coding sequence, and a terminator; and
B) a second vector comprising:
(i) from 5′ to 3′, and operably linked:
a second promoter,
a nucleotide sequence encoding a C-terminal fragment of the intein;
a nucleotide sequence encoding a C-terminal fragment of the selectable marker protein; and
a second terminator;
wherein the nucleotide sequence encoding the C-terminal fragment of the intein is linked, in frame, to the nucleotide sequence encoding the C-terminal fragment of the selectable marker protein;
and
(ii) a second gene of interest, wherein the second gene of interest comprises a promoter, a coding sequence, and a terminator;
wherein upon expression in a plant cell, the N-terminal fragment and the C-terminal fragment of the intein join the N-terminal fragment and the C-terminal fragment of the selectable marker protein to form a peptide bond.

2. The system of claim 1, wherein the first promoter and the second promoter are each an inducible or constitutive promoter.

3. The system of claim 1 or 2, wherein selectable marker protein is a protein that produces a visible signal.

4. The system of claim 3, wherein the visible signal is a red pigment or fluorescent signal.

5. The system of claim 4, wherein the selectable marker protein is RUBY or eYGFPuv.

6. The system of claim 5, wherein the selectable marker protein is RUBY, and the split between the N-terminal fragment and the C-terminal fragment of RUBY occurs at an amino acid position within the first 240 amino acids of SEQ ID NO: 1.

7. The system of claim 5, wherein the selectable marker protein is RUBY, and the split between the N-terminal fragment and the C-terminal fragment of RUBY occurs at amino acid position L231:C232 of SEQ ID NO: 1.

8. The system of claim 5, wherein the selectable marker protein is eYGFPuv, and the split between the N-terminal fragment and the C-terminal fragment of eYGFPuv occurs at an amino acid position within the first 75 amino acids of SEQ ID NO: 3.

9. The system of claim 8, wherein the selectable marker protein is eYGFPuv, and the split between the N-terminal fragment and the C-terminal fragment of eYGFPuv occurs at amino acid position T52:C53 of SEQ ID NO: 3.

10. The system of claim 1 or 2, wherein the selected marker protein is a protein encoded by an antibiotic resistance gene.

11. The system of claim 6, wherein the antibiotic resistance gene is a kanamycin or hygromycin resistance gene.

12. The system of claim 11, wherein the selectable marker protein is a protein encoded by kanamycin resistance gene, and the split between the N-terminal fragment and the C-terminal fragment of the protein occurs at an amino acid position within the first 200 amino acids of SEQ ID NO: 5.

13. The system of claim 11, wherein the selectable marker protein is a protein encoded by kanamycin resistance gene, and the split between the N-terminal fragment and the C-terminal fragment of the protein occurs at amino acid position T131:C132 or A192:C193 of SEQ ID NO: 5.

14. The system of claim 11, wherein the selectable marker protein is a protein encoded by hygromycin resistance gene, and the split between the N-terminal fragment and the C-terminal fragment of the protein occurs at an amino acid position within the first 100 amino acids of SEQ ID NO: 7.

15. The system of claim 14, wherein the selectable marker protein is a protein encoded by the hygromycin resistance gene, and the split between the N-terminal fragment and the C-terminal fragment of the protein occurs at amino acid position S52:C53 or Y89:C90 of SEQ ID NO: 7.

16. The system of any one of claims 1-15, wherein the intein is NpuDnaE.

17. The system of claim 16, wherein the split between the N-terminal fragment and the C-terminal fragment of NpuDnaE occurs at amino acid position within the first 110 amino acids.

18. The system of claim 16, wherein the split between the N-terminal fragment and the C-terminal fragment of NpuDnaE occurs at amino acid position N102:I103.

19. The system of any one of claims 1-18, wherein the plant is an herbaceous or woody plant.

20. The system of claim 19, wherein the herbaceous plant is selected from the group comprising Arabidopsis thaliana, Brassica rapa, Glycine max, Nicotiana benthamiana, Oryza sativa, Solanum lycopersicum, Solanum tuberosum, Panicum virgatum, Sorghum bicolor, and Zea mays.

21. The system of claim 19, wherein the woody plant is selected from the group comprising Citrus sinensis, Eucalyptus grandis, Malus domestica, Populus tremula x P. alba INRA 717-1B4, Prunus persica, and Vitis vinifera.

22. A method of co-transforming plant cells, the method comprising delivering DNA vectors into a plant cell:
A) a first vector comprising:
(i) from 5′ to 3′, and operably linked:
a first promoter,
a nucleotide sequence encoding an N-terminal fragment of a selectable marker protein; and
a nucleotide sequence encoding an N-terminal fragment of an intein;
wherein the nucleotide sequence encoding the N-terminal fragment of the selectable marker protein is linked, in frame, to the nucleotide sequence encoding the N-terminal fragment of the intein;
a first terminator;
and
(ii) a first gene of interest, wherein the first gene of interest comprises a promoter, a coding sequence, and a terminator; and
B) a second vector comprising:
(i) from 5′ to 3′, and operably linked:
a second promoter,
a nucleotide sequence encoding a C-terminal fragment of the intein; and
a nucleotide sequence encoding a C-terminal fragment of the selectable marker protein;
wherein the nucleotide sequence encoding the C-terminal fragment of the intein is linked, in frame, to the nucleotide sequence encoding the C-terminal fragment of the selectable marker protein;
a second terminator;
and
(ii) a second gene of interest, wherein the first gene of interest comprises a promoter, a coding sequence, and a terminator;
wherein upon expression in a plant cell, the N-terminal fragment and the C-terminal fragment of the intein join the N-terminal fragment and the C-terminal fragment of the selectable marker protein to form a peptide bond.

23. The method of claim 22, wherein the first promoter and the second promoter are each an inducible or constitutive promoter.

24. The method of claim 22 or 23, wherein the selectable marker protein is a protein that produces a visible signal.

25. The method of claim 24, wherein the visible signal is a red pigment or fluorescent signal.

26. The method of claim 25, wherein the selectable marker protein is RUBY or e YGFPuv.

27. The method of claim 26, wherein the selectable marker protein is RUBY, and the split between the N-terminal fragment and the C-terminal fragment of RUBY occurs at an amino acid position within the first 240 amino acids of SEQ ID NO: 1.

28. The method of claim 26, wherein the selectable marker protein is RUBY, and the split between the N-terminal fragment and the C-terminal fragment of RUBY occurs at amino acid position L231:C232 of SEQ ID NO: 1.

29. The method of claim 26, wherein the selectable marker protein is eYGFPuv, and the split between the N-terminal fragment and the C-terminal fragment of eYGFPuv occurs at an amino acid position within the first 75 amino acids of SEQ ID NO: 3.

30. The method of claim 26, wherein the selectable marker protein is eYGFPuv, and the split between the N-terminal fragment and the C-terminal fragment of eYGFPuv occurs at amino acid position T52:C53 of SEQ ID NO: 3.

31. The method of claim 22 or 23, wherein the selected marker protein is a protein encoded by an antibiotic resistance gene.

32. The method of claim 31, wherein the antibiotic resistance gene is a kanamycin or hygromycin resistance gene.

33. The method of claim 32, wherein the selectable marker protein is a protein encoded by kanamycin resistance gene, and the split between the N-terminal fragment and the C-terminal fragment of the protein occurs at an amino acid position within the first 200 amino acids of SEQ ID NO: 5.

34. The method of claim 33, wherein the selectable marker protein is a protein encoded by kanamycin resistance gene, and the split between the N-terminal fragment and the C-terminal fragment of the protein occurs at amino acid position T131:C132 or A192:C193 of SEQ ID NO: 5.

35. The method of claim 32, wherein the selectable marker protein is a protein encoded by hygromycin resistance gene, and the split between the N-terminal fragment and the C-terminal fragment of the protein occurs at an amino acid position within the first 100 amino acids of SEQ ID NO: 7.

37. The method of claim 35, wherein the selectable marker protein is a protein encoded by the hygromycin resistance gene, and the split between the N-terminal fragment and the C-terminal fragment of the protein occurs at amino acid position S52:C53 or Y89:C90 of SEQ ID NO: 7.28.

38. The method of any one of claims 22-37, wherein the intein is NpuDnaE.

39. The method of claim 38, wherein the split between the N-terminal fragment and the C-terminal fragment of NpuDnaE occurs at amino acid position within the first 110 amino acids.

40. The method of claim 39, wherein the split between the N-terminal fragment and the C-terminal fragment of NpuDnaE occurs at amino acid position N102:I103.

41. The method of any one of claims 22-40, wherein the plant is an herbaceous or woody plant.

42. The method of claim 41, wherein the herbaceous plant is selected from the group comprising Arabidopsis thaliana, Brassica rapa, Glycine max, Nicotiana benthamiana, Oryza sativa, Solanum lycopersicum, Solanum tuberosum, Panicum virgatum, Sorghum bicolor, and Zea mays.

43. The method of claim 41, wherein the woody plant is selected from the group comprising Citrus sinensis, Eucalyptus grandis, Malus domestica, Populus tremula x P. alba INRA 717-1B4, Prunus persica, and Vitis vinifera.
